(19) United States
(12) Patent Application Publication
(10) Pub. No.: US 2022/0102647 A1
(43) Pub. Date: Mar. 31, 2022

(54) Composition, Organic Semiconductor Layer and Electronic Device

(71) Applicants: Novaled GmbH, Dresden (DE); Samsung SDI Co., Ltd., Gyeonggi-do (KR)

(72) Inventors: Elena Galan, Dresden (DE); Benjamin Schulze, Dresden (DE); Francois Cardinali, Dresden (DE); Hyungsun Kim, Gyeonggi-do (KR); Seungjae Lee, Gyeonggi-do (KR)

(21) Appl. No.: 17/426,944

(22) PCT Filed: Jan. 30, 2020
(86) PCT No.: PCT/EP2020/052300

§ 371 Date: Jul. 29, 2021

(30) Foreign Application Priority Data
Jan. 30, 2019 (EP) 19154592.0

(51) Int. Cl. H01L 51/00; C09K 11/02; C07D 405/14

(52) U.S. Cl. CPC H01L 51/0067; C09K 11/02; H01L 51/5016; H01L 51/0072; H01L 51/0073; C07D 405/14

(57) ABSTRACT

The present invention relates to a composition, in particular to an organic semiconductor layer comprising a composition, suitable for use as an organic semiconductor layer for electronic devices, and a method of manufacturing the same, wherein the composition comprises: a) a compound of formula 1; and b) at least one organic metal complex, wherein the metal of the organic metal complex is selected from the group comprising alkali, alkaline earth or rare earth metal.

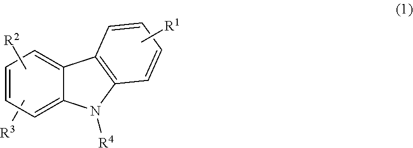

### TECHNICAL FIELD

The present invention relates to a composition, in particular to an organic semiconductor layer comprising the composition, suitable for use as an organic semiconductor layer for electronic devices, and a method of manufacturing the same.

### BACKGROUND ART

Organic electronic devices, such as organic light-emitting diodes OLEDs, which are self-emitting devices, have a wide viewing angle, excellent contrast, quick response, high brightness, excellent operating voltage characteristics, and color reproduction. A typical OLED comprises an anode, a hole transport layer HTL, an emission layer EML, an electron transport layer ETL, and a cathode, which are sequentially stacked on a substrate. In this regard, the HTL, the EML, and the ETL are thin films formed from organic compounds.

When a voltage is applied to the anode and the cathode, holes injected from the anode move to the EML, via the HTL, and electrons injected from the cathode move to the EML, via the ETL. The holes and electrons recombine in the EML to generate excitons. When the excitons drop from an excited state to a ground state, light is emitted. The injection and flow of holes and electrons should be balanced, so that an OLED having the above-described structure has excellent efficiency and/or a long lifetime.

Performance of an organic light emitting diode may be affected by characteristics of the organic semiconductor layer, and among them, may be affected by characteristics of an organic material of the organic semiconductor layer.

Particularly, development of an organic semiconductor layer being capable of increasing electron mobility and simultaneously increasing electrochemical stability is needed so that the organic electronic device, such as an organic light emitting diode, may be applied to a large-size flat panel display.

Further, development of an organic semiconductor layer being capable to have an extended life span at higher current density and thereby at higher brightness is needed.

There remains a need to improve performance of organic semiconductor materials, organic semiconductor layers, as well as organic electronic devices thereof, in particular to achieve increased lifetime through improving the characteristics of the compounds comprised therein.

### DISCLOSURE

An aspect of the present invention provides a composition comprising:
a) a compound of formula 1:

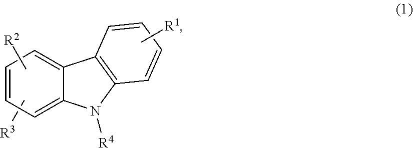

wherein
R¹ and R² are independently selected from H, D, CN, halogen, C₁ to C₁₂ alkyl, C₁ to C₁₂ alkoxy, substituted or unsubstituted C₆ to C₁₈ aryl, substituted or unsubstituted C₃ to C₂₄ heteroaryl,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
R³ or R⁴ has the general formula 2:

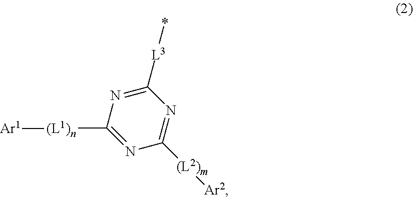

wherein
the asterisk symbol “*” represents the binding position of the group R³ or R⁴;
L¹, L² and L³ are independently selected from substituted or unsubstituted C₆ to C₁₃ arylene or substituted or unsubstituted C₃ to C₁₂ heteroarylene,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen;
n is 1 or 0;
m is 1 or 0;
Ar¹ is selected from substituted or unsubstituted C₅ to C₂₄ heteroaryl, preferably the C₅ to C₂₄ heteroaryl of the substituted or unsubstituted C₅ to C₂₄ heteroaryl of Ar¹ or the substituted or unsubstituted C₅ to C₂₄ heteroaryl of Ar¹ comprises at least one heteroaryl ring, at least one heteroaryl ring and at least one non-heteroaryl ring, at least two heteroaryl rings and at least one non-heteroaryl ring, at least three heteroaryl rings and at least one non-heteroaryl ring, at least one heteroaryl ring and at least two non-heteroaryl rings, or the substituted or unsubstituted heteroaryl are fused rings,
   wherein the substituents are selected from C₁ to C₁₂ alkyl, perhalogenated C₁ to C₁₂ alkyl, C₁ to C₁₂ alkoxy, perhalogenated C₁ to C₂₄ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
   wherein Ar¹ is free of a spiro-group;
Ar² has the general formula 3:

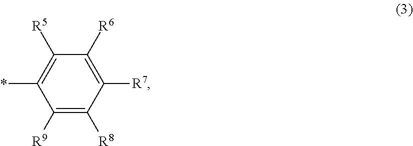

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, substituted or unsubstituted C₆ to C₁₂ aryl, substituted or unsubstituted C₃ to C₁₂ heteroaryl,
   wherein the substituted aryl ring or substituted heteroaryl ring is substituted with one or more C₁ to C₁₂ alkyl, C₆ to C₁₂ aryl or C₃ to C₁₂ heteroaryl groups, wherein the substituents are bonded by a single bond to the substituted aryl ring or substituted heteroaryl ring;
or
Ar² is selected from a fused ring system comprising one to four substituted or unsubstituted 6 membered aryl rings and zero to two substituted or unsubstituted 5 to 7 membered heteroaryl rings,
   wherein the substituted aryl ring is substituted with C₁ to C₁₂ alkyl groups and the substituted heteroaryl ring is substituted with one or more C₁ to C₁₂ alkyl, C₆ to C₁₂ aryl or C₃ to C₁₂ heteroaryl groups;
with the provision that:
i) R³ is formula 2 and R⁴ is selected from C₁ to C₁₂ alkyl, substituted or unsubstituted C₆ to C₁₈ aryl, substituted or unsubstituted C₃ to C₂₄ heteroaryl,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
   or
ii) R⁴ is formula 2 and R³ is H or D; and
b) at least one organic metal complex, wherein the metal of the organic metal complex is selected from the group comprising alkali, alkaline earth or rare earth metal.

Hetero atoms, if not otherwise stated, can be individually selected from N, O, S, B, Si, P, Se, preferably from N, O and S and more preferred is N.

According to one embodiment the substituted or unsubstituted C₅ to C₂₄ heteroaryl of Ar¹ may be selected from or may comprise at least one substituted or unsubstituted heteroaryl ring, at least one substituted or unsubstituted heteroaryl ring and at least one substituted or unsubstituted non-heteroaryl ring, at least two substituted or unsubstituted heteroaryl rings and at least one substituted or unsubstituted non-heteroaryl ring, at least three substituted or unsubstituted heteroaryl rings and at least one substituted or unsubstituted non-heteroaryl ring, at least one substituted or unsubstituted heteroaryl ring and at least two substituted or unsubstituted non-heteroaryl rings, or the substituted or unsubstituted C₅ to C₂₄ heteroaryl heteroaryl are fused rings.

According to one embodiment the C₅ to C₂₄ heteroaryl of the substituted or unsubstituted C₅ to C₂₄ heteroaryl of Ar¹ may be selected from or may comprise at least one substituted or unsubstituted heteroaryl ring, at least one substituted or unsubstituted heteroaryl ring and at least one substituted or unsubstituted non-heteroaryl ring, at least two substituted or unsubstituted heteroaryl rings and at least one substituted or unsubstituted non-heteroaryl ring, at least three substituted or unsubstituted heteroaryl rings and at least one substituted or unsubstituted non-heteroaryl ring, at least one substituted or unsubstituted heteroaryl ring and at least two substituted or unsubstituted non-heteroaryl rings, or the substituted or unsubstituted C₅ to C₂₄ heteroaryl heteroaryl are fused rings.

According to one embodiment, the composition comprising:
a) a compound of formula 1:

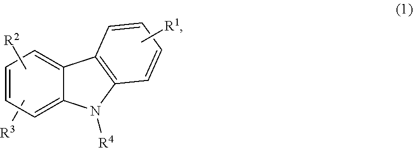

wherein
R¹ and R² are independently selected from H, D, CN, halogen, C₁ to C₁₂ alkyl, C₁ to C₁₂ alkoxy, substituted or unsubstituted C₆ to C₁₈ aryl comprising 1 to 3 of a 6-member aryl ring, substituted or unsubstituted C₃ to C₂₄ heteroaryl comprising 1 to 4 of a 6-member heteroaryl ring or at least one 6-member heteroaryl ring and 1 to 3 of a 6-member aryl ring, wherein rings can be fused,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
R³ or R⁴ has the general formula 2:

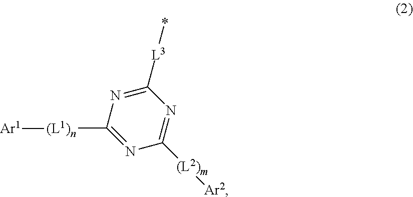

wherein
the asterisk symbol “*” represents the binding position of the group R³ or R⁴;
L¹, L² and L³ are independently selected from substituted or unsubstituted C₆ to C₁₃ arylene comprising 1 to 3 of a 6-member aryl ring, or substituted or unsubstituted C₃ to C₁₂ heteroarylene comprising 1 to 2 of a 6-member heteroaryl ring or one 6-member heteroaryl ring and one 6-member aryl ring, wherein rings thereof can be fused,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen;
n is 1 or 0;
m is 1 or 0;
Ar¹ is selected from substituted or unsubstituted C₅ to C₂₄ heteroaryl comprising at least 1 to 3 of a heteroaryl ring and 1 to 4 of an aryl ring, preferably at least 1 to 2 of a heteroaryl ring and 1 to 4 of an aryl ring, more preferred at least 1 to 2 of a 5-member heteroaryl ring and 1 to 2 of an 6-member aryl ring and in addition preferred one 5-member heteroaryl ring and 1 to 4 of an 6-member aryl ring, wherein rings thereof can be fused,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
   wherein Ar¹ is free of a spiro-group;
Ar² has the general formula 3:

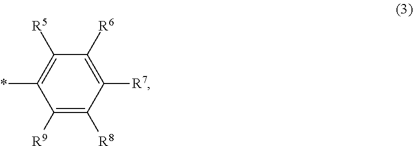

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, substituted or unsubstituted C₆ to C₁₂ aryl comprising 1 to 2 of a 6-member aryl ring, substituted or unsubstituted C₃ to C₁₂ heteroaryl comprising 1 to 2 of a heteroaryl ring or at least one heteroaryl ring and one 6-member aryl ring, wherein rings thereof can be fused,
   wherein the substituted aryl ring or substituted heteroaryl ring is substituted with one or more C₁ to C₁₂ alkyl, C₆ to C₁₂ aryl or C₃ to C₁₂ heteroaryl groups, wherein the substituents are bonded by a single bond to the substituted aryl ring or substituted heteroaryl ring;
or
Ar² is selected from a fused ring system comprising one to four substituted or unsubstituted 6 membered aryl rings and zero to two substituted or unsubstituted 5 to 7 membered heteroaryl rings, wherein rings thereof can be fused,
   wherein the substituted aryl ring is substituted with C₁ to C₁₂ alkyl groups and the substituted heteroaryl ring is substituted with one or more C₁ to C₁₂ alkyl, C₆ to C₁₂ aryl or C₃ to C₁₂ heteroaryl groups;
with the provision that:
   i) R³ is formula 2 and R⁴ is selected from C₁ to C₁₂ alkyl, substituted or unsubstituted C₆ to C₁₈ aryl, substituted or unsubstituted C₃ to C₂₄ heteroaryl,
      wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
         wherein R′ and R″ are independently selected from C₁ to C₁₆ alkyl, C₆ to C₂₄ aryl, C₃ to C₂₄ heteroaryl;
   or
   ii) R⁴ is formula 2 and R³ is H or D; and
b) at least one organic metal complex, wherein the metal of the organic metal complex is selected from the group comprising alkali, alkaline earth or rare earth metal

If not otherwise stated H can represent hydrogen or deuterium.

The composition can be an organic semiconductor.

According to one embodiment, the composition comprising:
a) a compound of formula 1:

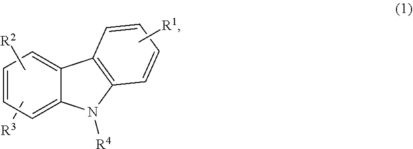

wherein
R¹ and R² are independently selected from H, D, CN, halogen, C₁ to C₁₂ alkyl, C₁ to C₁₂ alkoxy, substituted or unsubstituted C₆ to C₁₈ aryl, substituted or unsubstituted C₃ to C₂₄ heteroaryl,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
R³ or R⁴ has the general formula 2:

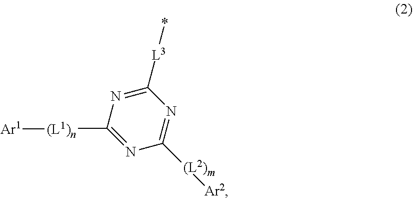

wherein
the asterisk symbol “*” represents the binding position of the group R³ or R⁴;
L¹, L² and L³ are independently selected from substituted or unsubstituted C₆ to C₁₃ arylene or substituted or unsubstituted C₃ to C₁₂ heteroarylene,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen;
n is 1 or 0;
m is 1 or 0;
Ar¹ is selected from substituted or unsubstituted C₅ to C₂₄ heteroaryl, wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR
   wherein R′ and R″ are independently selected from C₁ to C₁₆ alkyl, C₆ to C₂₄ aryl, C₃ to C₂₄ heteroaryl;
Ar² has the general formula 3:

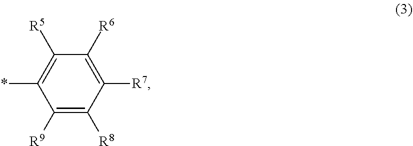

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, substituted or unsubstituted C₆ to C₁₂ aryl, substituted or unsubstituted C₃ to C₁₂ heteroaryl,
   wherein the substituted aryl ring or substituted heteroaryl ring is substituted with one or more C₁ to C₁₂ alkyl, C₆ to C₁₂ aryl or C₃ to C₁₂ heteroaryl groups, wherein the substituents are bonded by a single bond to the substituted aryl ring or substituted heteroaryl ring;
or
Ar² is selected from a fused ring system comprising one to four substituted or unsubstituted 6 membered aryl rings and zero to two substituted or unsubstituted 5 to 7 membered heteroaryl rings,
   wherein the substituted aryl ring is substituted with C₁ to C₁₂ alkyl groups and the substituted heteroaryl ring is substituted with one or more C₁ to C₁₂ alkyl, C₆ to C₁₂ aryl or C₃ to C₁₂ heteroaryl groups;
with the provision that:
i) R³ is formula 2 and R⁴ is selected from C₁ to C₁₂ alkyl, substituted or unsubstituted C₆ to C₁₈ aryl, substituted or unsubstituted C₃ to C₂₄ heteroaryl,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
   or
ii) R⁴ is formula 2 and R³ is H or D;
wherein the compound of formula 1 is free of a spiro-group; and
b) at least one organic metal complex, wherein the metal of the organic metal complex is selected from the group comprising alkali, alkaline earth or rare earth metal.

According to one embodiment, the composition comprising:
a) a compound of formula 1:

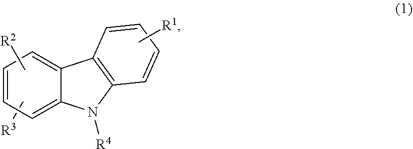

wherein
R¹ and R² are independently selected from H, D, CN, halogen, C₁ to C₁₂ alkyl, C₁ to C₁₂ alkoxy, substituted or unsubstituted C₆ to C₁₈ aryl, substituted or unsubstituted C₃ to C₂₄ heteroaryl,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
R³ or R⁴ has the general formula 2:

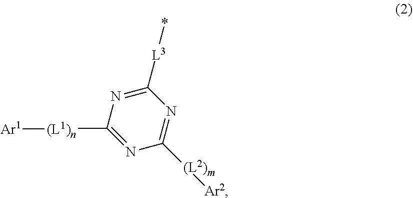

wherein
the asterisk symbol “*” represents the binding position of the group R³ or R⁴;
L¹, L² and L³ are independently selected from substituted or unsubstituted C₆ to C₁₃ arylene or substituted or unsubstituted C₃ to C₁₂ heteroarylene,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen;
n is 1 or 0;
m is 1 or 0;
Ar¹ is selected from substituted or unsubstituted C₅ to C₂₄ heteroaryl, wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
   wherein R′ and R″ are independently selected from C₁ to C₁₆ alkyl, C₆ to C₂₄ aryl, C₃ to C₂₄ heteroaryl;
Ar² has the general formula 3:

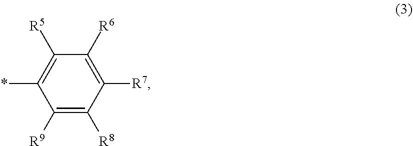

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, unsubstituted C₆ to C₁₂ aryl, or unsubstituted C₃ to C₁₂ heteroaryl;
or
Ar² is selected from a fused ring system comprising one to four unsubstituted 6 membered aryl rings and zero to two unsubstituted 5 to 7 membered heteroaryl rings;
with the provision that:
i) R³ is formula 2 and R⁴ is selected from C₁ to C₁₂ alkyl, substituted or unsubstituted C₆ to C₁₈ aryl, substituted or unsubstituted C₃ to C₂₄ heteroaryl,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
      wherein R′ and R″ are independently selected from C₁ to C₁₆ alkyl, C₆ to C₂₄ aryl, C₃ to C₂₄ heteroaryl;
   or
ii) R⁴ is formula 2 and R³ is H or D;
wherein the compound of formula 1 is free of a spiro-group; and
b) at least one organic metal complex, wherein the metal of the organic metal complex is selected from the group comprising alkali, alkaline earth or rare earth metal.

According to one embodiment, the composition comprising:
a) a compound of formula 1:

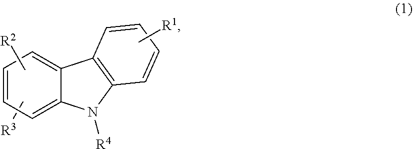

wherein
R¹ and R² are independently selected from H, D, CN, halogen, C₁ to C₁₂ alkyl, C₁ to C₁₂ alkoxy, unsubstituted C₆ to C₁₈ aryl, unsubstituted C₃ to C₂₄ heteroaryl;
R³ or R⁴ has the general formula 2;

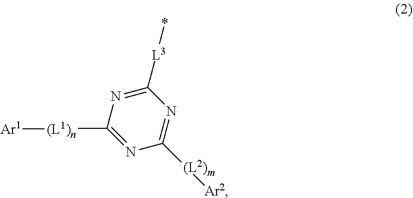

wherein
the asterisk symbol “*” represents the binding position of the group R³ or R⁴;
L¹, L² and L³ are independently selected from unsubstituted C₆ to C₁₃ arylene or unsubstituted C₃ to C₁₂ heteroarylene;
n is 1 or 0;
m is 1 or 0;
Ar¹ is selected from unsubstituted C₅ to C₂₄ heteroaryl;
wherein Ar¹ is free of a spiro-group or the compound of formula 1 is free of a spiro-group;
Ar² has the general formula 3:

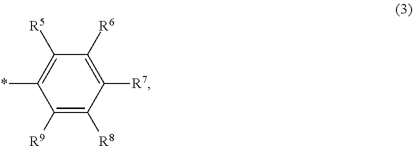

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, unsubstituted C₆ to C₁₂ aryl, unsubstituted C₃ to C₁₂ heteroaryl;
or
Ar² is selected from a fused ring system comprising one to four unsubstituted 6 membered aryl rings and zero to two unsubstituted 5 to 7 membered heteroaryl rings;
with the provision that:
iii) R³ is formula 2 and R⁴ is selected from C₁ to C₁₂ alkyl, unsubstituted C₆ to C₁₈ aryl, unsubstituted C₃ to C₂₄ heteroaryl;
   or
iv) R⁴ is formula 2 and R³ is H or D; and
b) at least one organic metal complex, wherein the metal of the organic metal complex is selected from the group comprising alkali, alkaline earth or rare earth metal.

According to another embodiment of the compound of formula 1, wherein the hetero atom of the C₃ to C₂₄ heteroaryl, C₃ to C₁₂ heteroaryl, C₃ to C₁₂ heteroaryl ene, may be selected from N, O or S.

According to another embodiment of the compound of formula 1, wherein the hetero atom of the C₃ to C₂₄ heteroaryl, C₃ to C₁₂ heteroaryl, C₃ to C₁₂ heteroaryl ene, may be selected from N or O.

According to another embodiment of the compound of formula 1, wherein R¹, R² may be independently selected from substituted or unsubstituted C₁ to C₁₆ alkyl, substituted or unsubstituted C₆ to C₁₂ aryl, substituted or unsubstituted C₃ to C₁₇ heteroaryl, wherein the substituents of the substituted C₆ to C₁₂ aryl and substituted C₃ to C₁₇ heteroarylene are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′, wherein R′ and R″ are independently selected from C₁ to C₁₆ alkyl, C₆ to C₂₄ aryl, C₃ to C₂₄ heteroaryl.

According to another embodiment of the compound of formula 1, wherein R¹, R² may be independently selected from H, D, unsubstituted C₆ to C₁₈ aryl, or unsubstituted C₃ to C₂₄ heteroaryl. According to another embodiment of the compound of formula 1, wherein R¹, R² may be preferably independently selected from H, D, or unsubstituted C₆ to C₁₈ aryl. According to another embodiment of the compound of formula 1, wherein R¹, R² may be further preferred independently selected from H, D, or unsubstituted C₆ aryl. According to another embodiment of the compound of formula 1, wherein R¹ may in addition preferred independently selected from H or D and R² is a unsubstituted C₆ aryl. According to another embodiment of the compound of formula 1, wherein R² is in addition preferred selected from H or D and R¹ is an unsubstituted C₆ aryl.

According to another embodiment of the compound of formula 1, wherein R¹, R² are independently selected from H, D, phenyl, biphenyl, terphenyl naphthyl, phenanthrenyl, pyridyl, quinolinyl, quinazolinyl.

According to another embodiment of the compound of formula 1, wherein R¹, R² are independently selected from H, D, phenyl, biphenyl, terphenyl, naphthyl, phenanthrenyl.

According to another embodiment of the compound of formula 1, wherein R¹, R² are independently selected from H, D, unsubstituted C₆ to C₁₂ aryl, unsubstituted C₃ to C₁₇ heteroaryl, and preferably from H, D, phenyl and biphenyl and more preferred from H, D, and phenyl.

According to another embodiment of the compound of formula 1, wherein Ar¹ may be selected from substituted or unsubstituted C₅ to C₂₄ heteroaryl, wherein the substituents of the substituted C₅ to C₂₄ heteroaryl may be selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′, wherein R′ and R″ are independently selected from C₁ to C₁₆ alkyl, C₆ to C₂₄ aryl, C₃ to C₂₄ heteroaryl.

According to another embodiment of the compound of formula 1, wherein Ar¹ may be selected from substituted or unsubstituted C₅ to C₁₈ heteroaryl, wherein the substituents of the substituted C₅ to C₁₈ heteroaryl may be selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′, wherein R′ and R″ are independently selected from C₁ to C₁₆ alkyl, C₆ to C₂₄ aryl, C₃ to C₂₄ heteroaryl.

According to another embodiment of the compound of formula 1, wherein Ar¹ may be selected from substituted or unsubstituted C₅ to C₁₈ heteroaryl, wherein the substituents of the substituted C₅ to C₁₈ heteroaryl may be selected from C₁ to C₁₂ alkyl, perhalogenated C₁ to C₁₂ alkyl, C₆ to C₂₄ aryl, perhalogenated C₆ to C₂₄ aryl, C₃ to C₂₄ heteroaryl, CN, halogen.

According to another embodiment of the compound of formula 1, wherein Ar¹ may be selected from substituted or unsubstituted C₅ to C₁₈ heteroaryl, wherein the substituents of the substituted C₅ to C₁₈ heteroaryl may be selected from C₁ to C₁₂ alkyl, C₆ to C₂₄ aryl, C₃ to C₂₄ heteroaryl.

According to another embodiment of the compound of formula 1, wherein Ar¹ may be selected from unsubstituted C₅ to C₂₄ heteroaryl.

According to another embodiment of the compound of formula 1, wherein Ar¹ may be selected from substituted or unsubstituted C₅ to C₂₄ heteroaryl comprising at least one heteroatom selected from O, S, Se or N, preferably the at least one heteroatom is selected from O, S, Se and in addition preferred the at least one heteroatom is O.

According to another embodiment of the compound of formula 1, wherein Ar¹ may be selected from unsubstituted C₅ to C₁₈ heteroaryl.

According to another embodiment of the compound of formula 1, wherein Ar¹ may have the general formula 4:

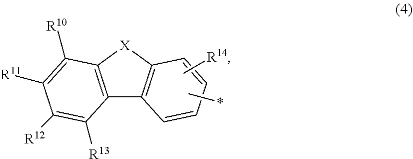

wherein the asterisk symbol “*” represents the binding position of the group Ar¹ to the moiety L¹;
X is selected from O, S, Se or NR¹⁵,
   wherein R¹⁵ is independently selected from C₁ to C₁₂ alkyl, perhalogenated C₁ to C₁₂ alkyl, C₆ to C₂₄ aryl, perhalogenated C₆ to C₂₄ aryl, C₃ to C₂₄ heteroaryl;
R¹⁰ to R¹⁴ are independently selected from H, D, CN, halogen, C₁ to C₁₂ alkyl, C₁ to C₁₂ alkoxy, C₆ to C₁₈ aryl or C₃ to C₂₄ heteroaryl;
wherein preferably R¹⁰ and R¹¹, R¹¹ and R¹² or R¹² and R¹³ form a 5 to 7 membered substituted or unsubstituted aryl ring, or a 5 to 7 membered substituted or unsubstituted heteroaryl ring, and in addition preferred R¹⁰ and R¹¹, R¹¹ and R¹² or R¹² and R¹³ form two fused 5 to 7 membered substituted or unsubstituted aryl rings, or two fused 5 to 7 membered substituted or unsubstituted heteroaryl rings,
   wherein the substituents are selected from C₁ to C₁₂ alkyl, C₆ to C₁₈ aryl or C₃ to C₂₄ heteroaryl groups.

According to another embodiment of the compound of formula 1, wherein X in general formula 4 can be selected from O, S or NR¹⁵, more preferred X is selected from O or S, and also preferred X is O.

According to another embodiment of the compound of formula 1, wherein X in general formula 4 can be selected from O, S or NR¹⁵, and R¹⁰ to R¹⁴ are H.

According to another embodiment of the compound of formula 1, wherein Ar¹ may be independently selected from the group comprising D1 to D16:

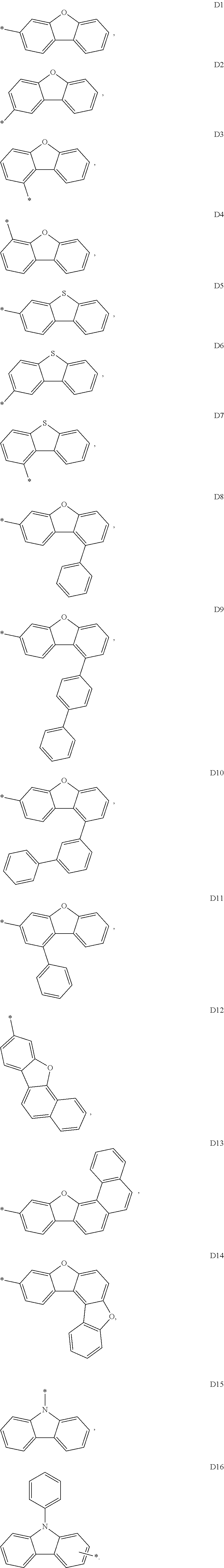

In another embodiment, Ar¹ may be selected from D1 to D14. In another embodiment, Ar¹ may be preferably selected from D1, D3, D5, D7, D8 to D14. In another embodiment, Ar¹ may be more preferably selected from D1 to D7. In another embodiment, Ar¹ may be more preferably selected from D1 or D3. In another embodiment, Ar¹ may be furthermore preferably selected from D1.

According to another embodiment of the compound of formula 1, wherein Ar² may have the general formula 3:

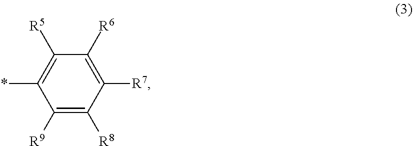

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, substituted or unsubstituted C₆ to C₁₂ aryl, substituted or unsubstituted C₃ to C₁₂ heteroaryl,
   wherein the substituted aryl ring or substituted heteroaryl ring is substituted with one or more C₁ to C₁₂ alkyl, C₆ to C₁₂ aryl or C₃ to C₁₂ heteroaryl groups,
   wherein the substituents are bonded by a single bond to the substituted aryl ring or substituted heteroaryl ring.

According to another embodiment of the compound of formula 1, wherein Ar² may have the general formula 3:

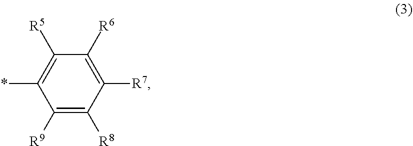

wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, substituted or unsubstituted C₆ to C₁₂ aryl, substituted or unsubstituted C₃ to C₁₂ heteroaryl,

wherein the substituted aryl ring or substituted heteroaryl ring is substituted with one or more C₁ to C₁₂ alkyl, C₆ to C₁₂ aryl or C₃ to C₁₂ heteroaryl groups,

wherein the substituents are bonded by a single bond to the substituted aryl ring or substituted heteroaryl ring; and wherein the group Ar² can be free of a fluorene group.

According to another embodiment of the compound of formula 1, wherein Ar² may be selected from a fused ring system comprising one to four substituted or unsubstituted 6 membered aryl rings and zero to two substituted or unsubstituted 5 to 7 membered heteroaryl rings,

wherein the substituted aryl ring is substituted with C₁ to C₁₂ alkyl groups and the

substituted heteroaryl ring is substituted with one or more C₁ to C₁₂ alkyl, C₆ to C₁₂ aryl or C₃ to C₁₂ heteroaryl groups;

and R³ is formula 2 and R⁴ is selected from C₁ to C₁₂ alkyl, substituted or unsubstituted C₆ to C₁₈ aryl, substituted or unsubstituted C₃ to C₂₄ heteroaryl,
wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
   wherein R′ and R″ are independently selected from C₁ to C₁₆ alkyl, C₆ to C₂₄ aryl, C₃ to C₂₄ heteroaryl.

According to another embodiment of the compound of formula 1, wherein Ar² may be selected from a fused ring system comprising one to four substituted or unsubstituted 6 membered aryl rings and zero to two substituted or unsubstituted 5 to 7 membered heteroaryl rings,
wherein the substituted aryl ring is substituted with C₁ to C₁₂ alkyl groups and the substituted heteroaryl ring is substituted with one or more C₁ to C₁₂ alkyl, C₆ to C₁₂ aryl or C₃ to C₁₂ heteroaryl groups;
   and R⁴ is formula 2 and R³ is H or D.

According to another embodiment of the compound of formula 1, wherein Ar² is free of a fluorene group.

According to another embodiment of the compound of formula 1, wherein Ar² can be selected from the group comprising E1 to E44:

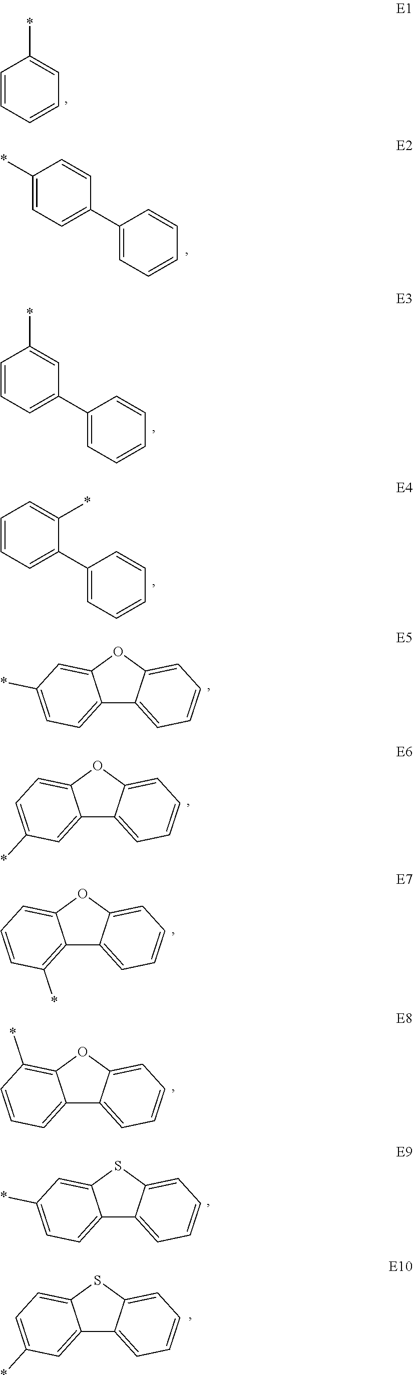

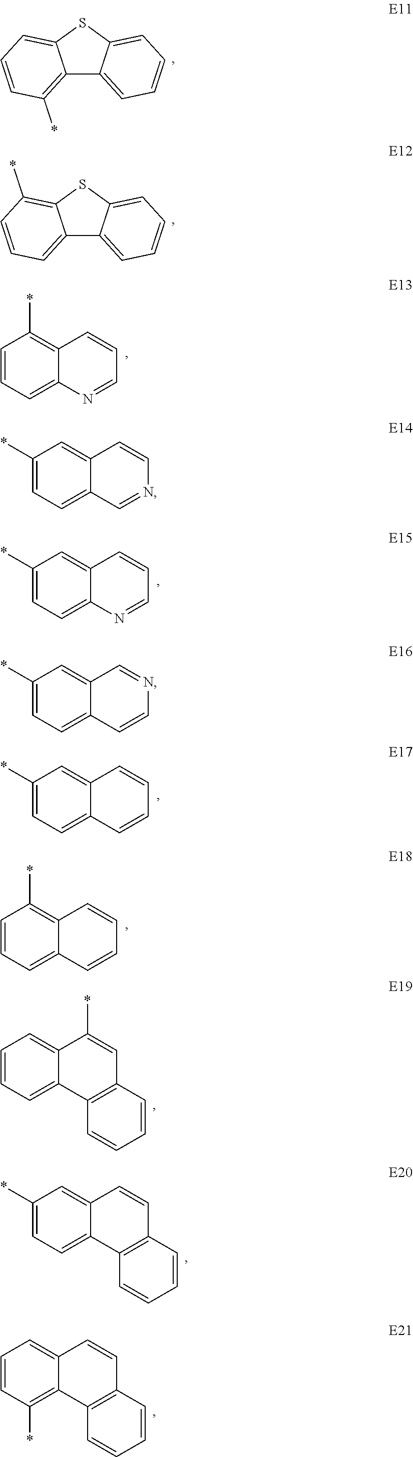

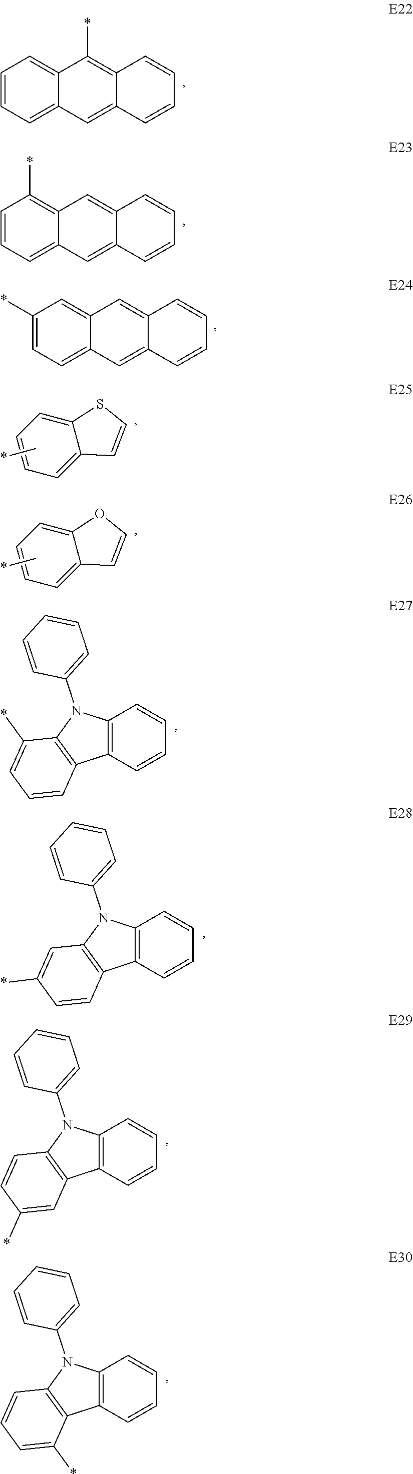

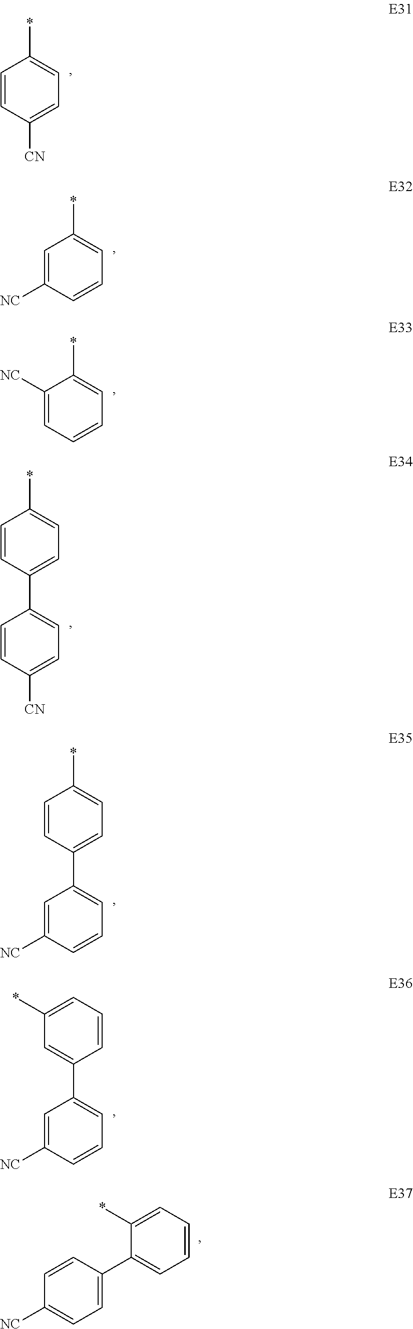

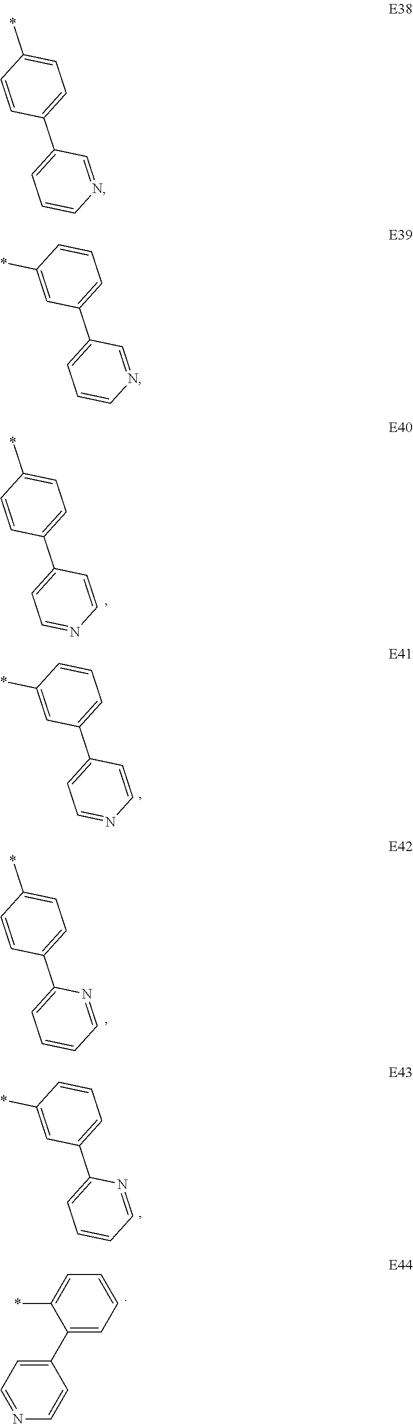

According to another embodiment of the compound of formula 1, wherein Ar² can be preferably selected from the group comprising E1 to E26 and E31 to E44. According to another embodiment of the compound of formula 1, wherein Ar² can be more preferably selected from the group comprising E1 to E12, E17 to E19 and E31 to E37. According to another embodiment of the compound of formula 1, wherein Ar² can be also preferred selected from the group comprising E1 to E12, E17 to E19 and E31 to E37. According to another embodiment of the compound of formula 1, wherein Ar² can be most preferred selected from the group comprising E1 to E4 and E17 to E18.

According to another embodiment of the compound of formula 1, wherein
L¹, L² and L³ are independently selected from substituted or unsubstituted C₆ to C₁₃ arylene or substituted or unsubstituted C₃ to C₁₂ heteroarylene,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen.

According to another embodiment of the compound of formula 1, wherein
L¹, L² and L³ are independently selected from substituted or unsubstituted C₆ arylene or substituted or unsubstituted C₃ to C₅ heteroarylene,
   wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen.

According to another embodiment of the compound of formula 1, wherein
L¹, L² and L³ are independently selected from unsubstituted C₆ to C₁₃ arylene or unsubstituted C₃ to C₁₂ heteroaryl ene, preferably independently selected from unsubstituted C₆ arylene or unsubstituted C₃ to C₅ heteroaryl ene.

According to another embodiment of the compound of formula 1, wherein
a) L¹, L² and/or L³ may be independently selected from the group comprising F1 to F11:

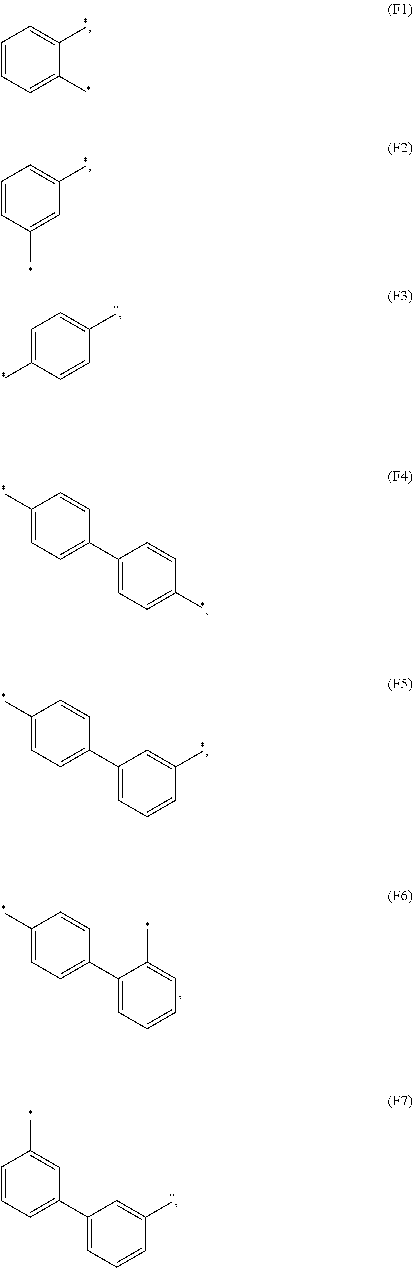

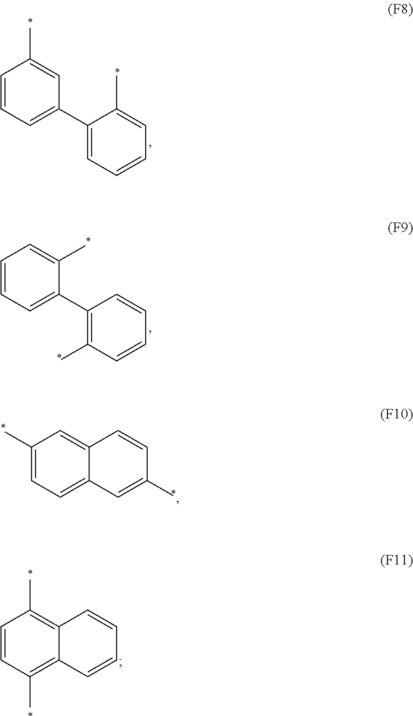

preferably L¹, L² and/or L³ can be selected from F2, F3, F4, F5 or F7, more preferably L¹, L² and/or L³ can be selected from F2, F3, F5 or F7, more preferred L¹, L² and/or L³ can be selected from F2 or F3; or
b) for L¹ n is 1 and for L² m is 0, L¹ may be selected from F1 to F11, preferably L¹ can be selected from F2, F3, F4, F5 or F7, more preferably L¹ can be selected from F2, F3, F5 or F7, more preferred L¹ can be selected from F2 or F3; or
c) for L¹ n is 0 and for L² m can be 1, L² is selected from F1 to F11, preferably L² can be selected from F2, F3, F4, F5 or F7, more preferably L² can be selected from F2, F3, F5 or F7, more preferred L² can be selected from F2 or F3; or
d) for L¹ n is 0 and for L² m is 0 and L³ may be selected from F1 to F11, preferably L³ can be selected from F2, F3, F4, F5 or F7, more preferably L³ can be selected from F2, F3, F5 or F7, more preferred L³ is selected from F2 or F3.

According to another embodiment of the compound of formula 1, wherein n=1 and m=0. According to another embodiment of the compound of formula 1, wherein n=0 and m=1. According to another more preferred embodiment of the compound of formula 1, wherein n=0 and m=0.

According to another embodiment of the compound of formula 1, wherein n=1 and m=0 and L³ is a substituted or unsubstituted phenylene or substituted or unsubstituted biphenylene group, wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen. According to another embodiment of the compound of formula 1, wherein n=0 and m=1 and L³ is a substituted or unsubstituted phenylene or substituted or unsubstituted biphenylene group, wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen. According to another more preferred embodiment of the compound of formula 1, wherein n=0 and m=0 and L³ is a substituted or unsubstituted phenylene or substituted or unsubstituted biphenylene group, wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen.

According to another embodiment of the compound of formula 1, wherein n=1 and m=0 and L³ is a unsubstituted phenylene or unsubstituted biphenylene group. According to another embodiment of the compound of formula 1, wherein n=0 and m=1 and L³ is a unsubstituted phenylene or unsubstituted biphenylene group. According to another more preferred embodiment of the compound of formula 1, wherein n=0 and m=0 and L³ is a unsubstituted phenylene or unsubstituted biphenylene group.

According to another embodiment of the compound of formula 1 may be selected from G1 to G31:

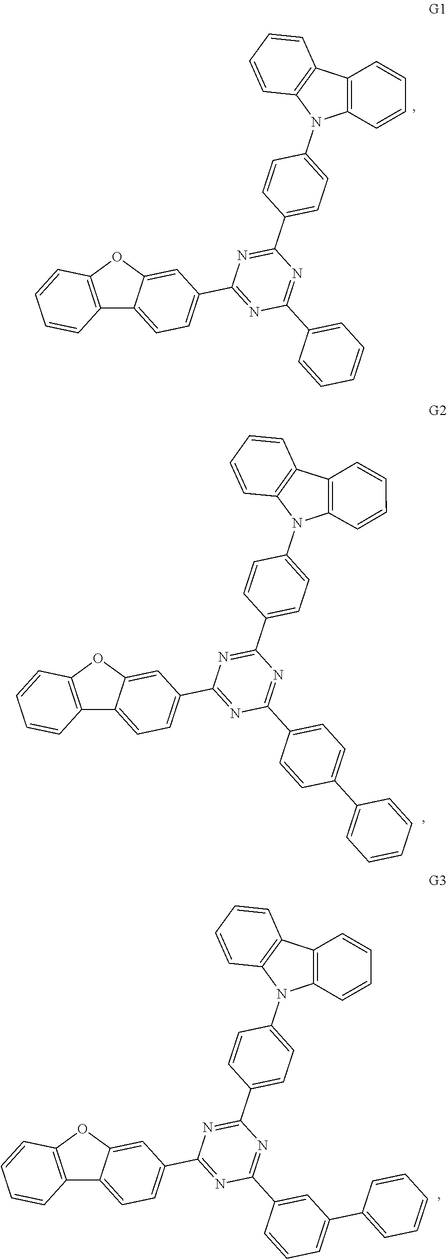 [SMILES: c1ccc(-c2nc(-c3ccc(-n4c5ccccc5c5ccccc54)cc3)nc(-c3ccc4c(c3)oc3ccccc34)n2)cc1 ; c1ccc(-c2ccc(-c3nc(-c4ccc(-n5c6ccccc6c6ccccc65)cc4)nc(-c4ccc5c(c4)oc4ccccc45)n3)cc2)cc1 ; c1ccc(-c2cccc(-c3nc(-c4ccc(-n5c6ccccc6c6ccccc65)cc4)nc(-c4ccc5c(c4)oc4ccccc45)n3)c2)cc1]

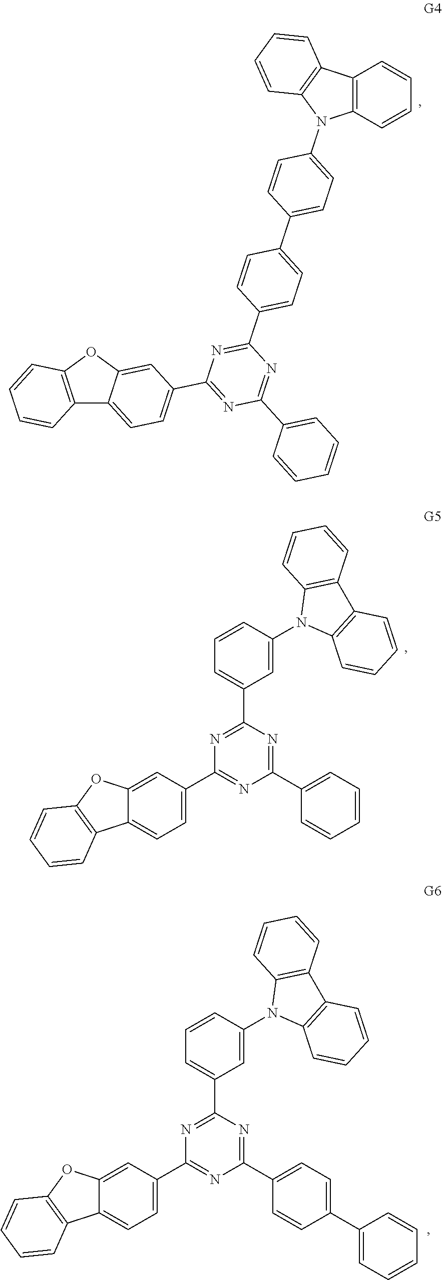 [SMILES: c1ccc(-c2nc(-c3ccc(-c4ccc(-n5c6ccccc6c6ccccc65)cc4)cc3)nc(-c3ccc4c(c3)oc3ccccc34)n2)cc1 ; c1ccc(-c2nc(-c3cccc(-n4c5ccccc5c5ccccc54)c3)nc(-c3ccc4c(c3)oc3ccccc34)n2)cc1 ; c1ccc(-c2ccc(-c3nc(-c4cccc(-n5c6ccccc6c6ccccc65)c4)nc(-c4ccc5c(c4)oc4ccccc45)n3)cc2)cc1]

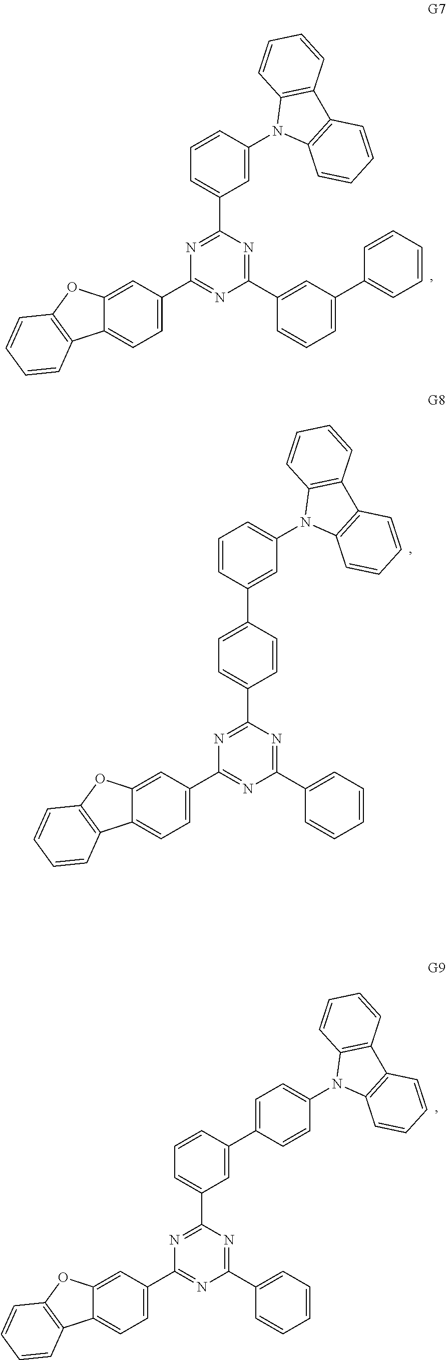 [SMILES: c1ccc(-c2cccc(-c3nc(-c4cccc(-n5c6ccccc6c6ccccc65)c4)nc(-c4ccc5c(c4)oc4ccccc45)n3)c2)cc1 ; c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2nc(-c3cccc(-c4ccc(-n5c6ccccc6c6ccccc65)cc4)c3)nc(-c3ccc4c(c3)oc3ccccc34)n2)cc1]

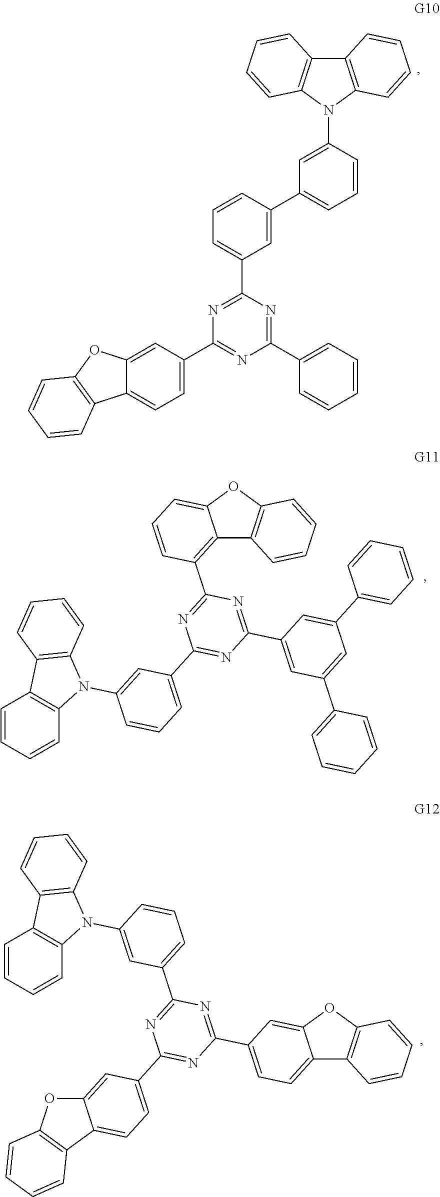 [SMILES: c1ccc(-c2nc(-c3cccc(-c4cccc(-n5c6ccccc6c6ccccc65)c4)c3)nc(-c3ccc4c(c3)oc3ccccc34)n2)cc1 ; c1ccc(-c2cc(-c3ccccc3)cc(-c3nc(-c4cccc(-n5c6ccccc6c6ccccc65)c4)nc(-c4cccc5oc6ccccc6c45)n3)c2)cc1 ; c1cc(-c2nc(-c3ccc4c(c3)oc3ccccc34)nc(-c3ccc4c(c3)oc3ccccc34)n2)cc(-n2c3ccccc3c3ccccc32)c1]

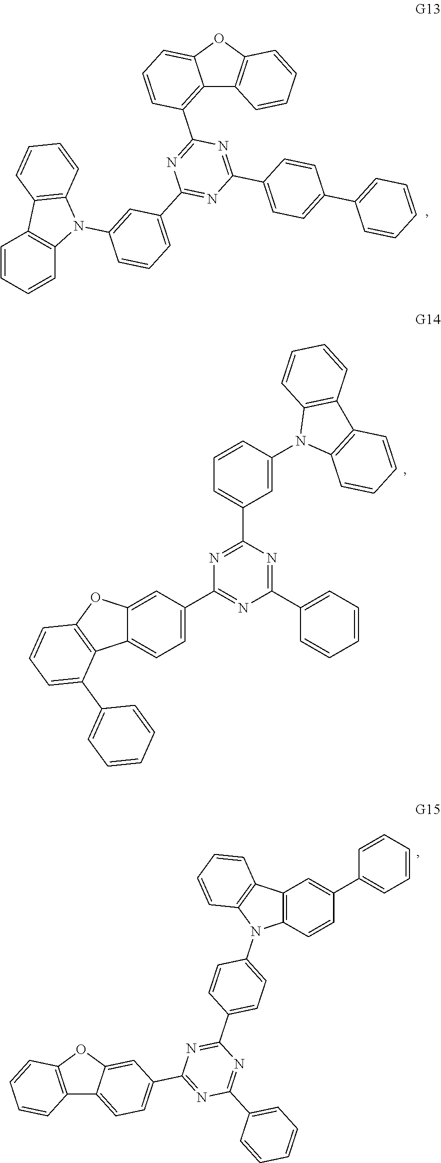 [SMILES: c1ccc(-c2ccc(-c3nc(-c4cccc(-n5c6ccccc6c6ccccc65)c4)nc(-c4cccc5oc6ccccc6c45)n3)cc2)cc1 ; c1ccc(-c2nc(-c3cccc(-n4c5ccccc5c5ccccc54)c3)nc(-c3ccc4c(c3)oc3cccc(-c5ccccc5)c34)n2)cc1 ; c1ccc(-c2ccc3c(c2)c2ccccc2n3-c2ccc(-c3nc(-c4ccccc4)nc(-c4ccc5c(c4)oc4ccccc45)n3)cc2)cc1]

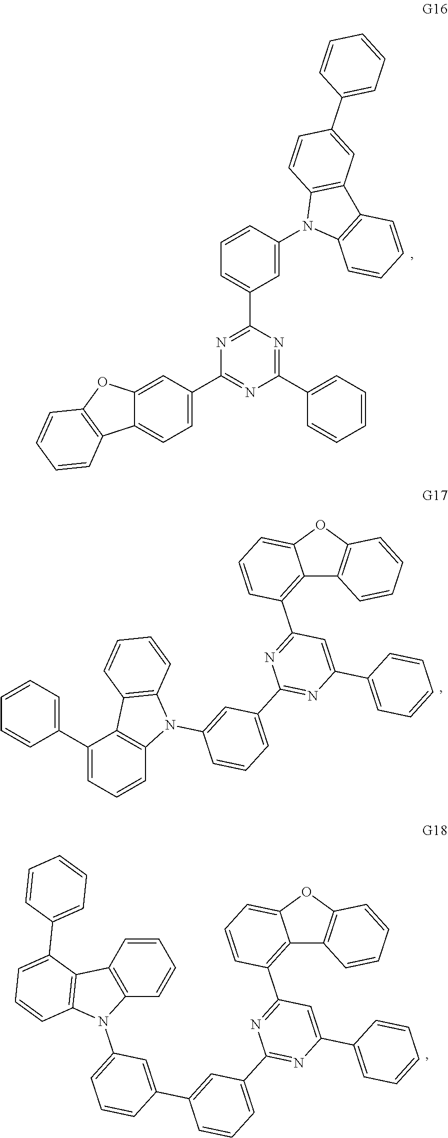 [SMILES: c1ccc(-c2ccc3c(c2)c2ccccc2n3-c2cccc(-c3nc(-c4ccccc4)nc(-c4ccc5c(c4)oc4ccccc45)n3)c2)cc1 ; c1ccc(-c2cc(-c3cccc4oc5ccccc5c34)nc(-c3cccc(-n4c5ccccc5c5c(-c6ccccc6)cccc54)c3)n2)cc1 ; c1ccc(-c2cc(-c3cccc4oc5ccccc5c34)nc(-c3cccc(-c4cccc(-n5c6ccccc6c6c(-c7ccccc7)cccc65)c4)c3)n2)cc1]

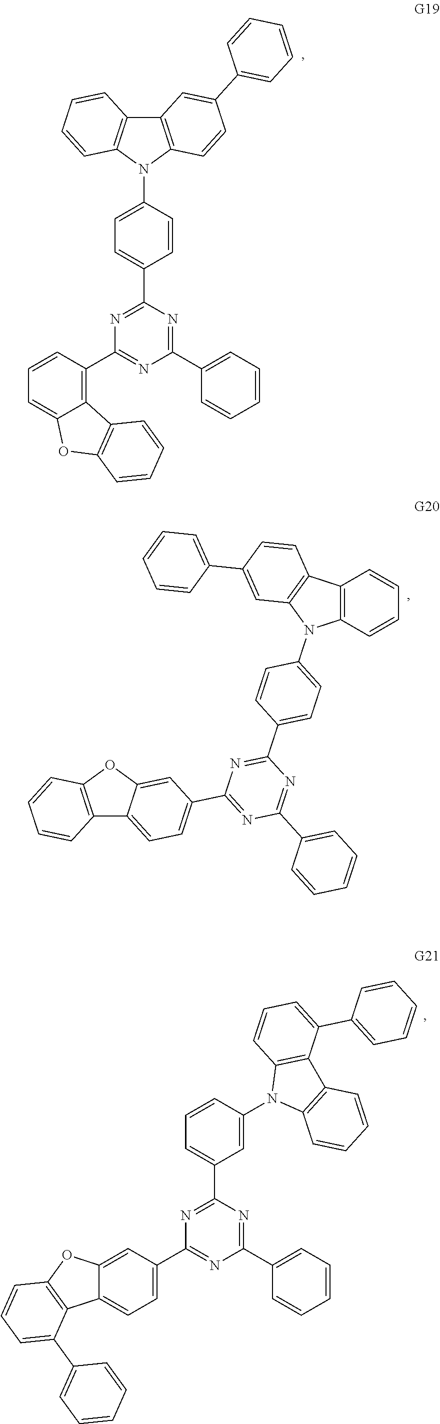 [SMILES: c1ccc(-c2ccc3c(c2)c2ccccc2n3-c2ccc(-c3nc(-c4ccccc4)nc(-c4cccc5oc6ccccc6c45)n3)cc2)cc1 ; c1ccc(-c2ccc3c4ccccc4n(-c4ccc(-c5nc(-c6ccccc6)nc(-c6ccc7c(c6)oc6ccccc67)n5)cc4)c3c2)cc1 ; c1ccc(-c2nc(-c3cccc(-n4c5ccccc5c5c(-c6ccccc6)cccc54)c3)nc(-c3ccc4c(c3)oc3cccc(-c5ccccc5)c34)n2)cc1]

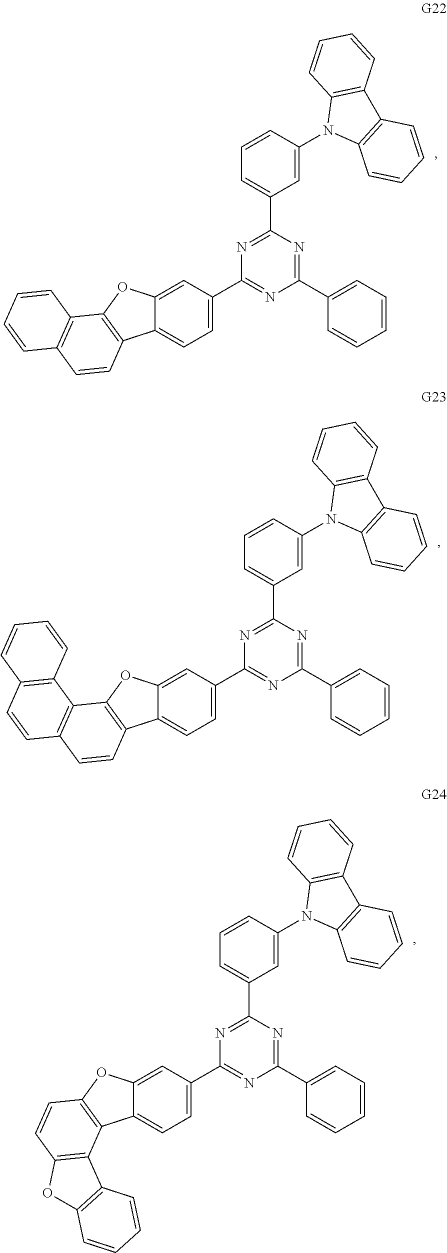 [SMILES: c1ccc(-c2nc(-c3cccc(-n4c5ccccc5c5ccccc54)c3)nc(-c3ccc4c(c3)oc3c5ccccc5ccc43)n2)cc1 ; c1ccc(-c2nc(-c3cccc(-n4c5ccccc5c5ccccc54)c3)nc(-c3ccc4c(c3)oc3c4ccc4ccc5ccccc5c43)n2)cc1 ; c1ccc(-c2nc(-c3cccc(-n4c5ccccc5c5ccccc54)c3)nc(-c3ccc4c(c3)oc3ccc5oc6ccccc6c5c34)n2)cc1]

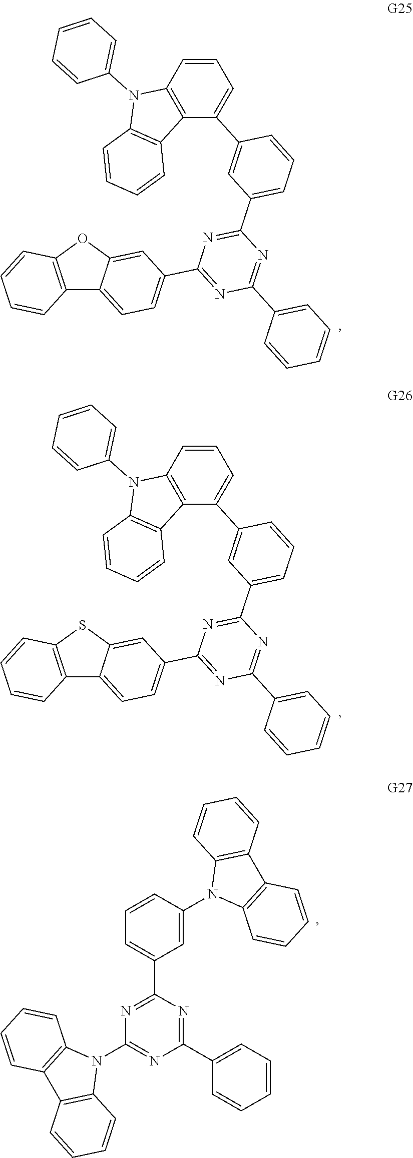 [SMILES: c1ccc(-c2nc(-c3cccc(-c4cccc5c4c4ccccc4n5-c4ccccc4)c3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)sc3ccccc34)n2)cc1 ; c1ccc(-c2nc(-c3cccc(-n4c5ccccc5c5ccccc54)c3)nc(-n3c4ccccc4c4ccccc43)n2)cc1]

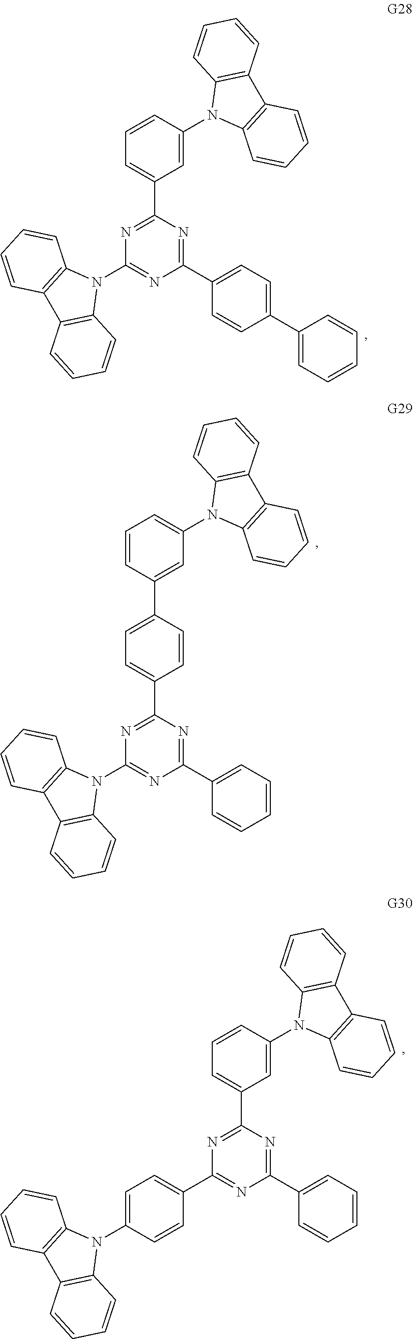 [SMILES: c1ccc(-c2ccc(-c3nc(-c4cccc(-n5c6ccccc6c6ccccc65)c4)nc(-n4c5ccccc5c5ccccc54)n3)cc2)cc1 ; c1ccc(-c2nc(-c3ccc(-c4cccc(-n5c6ccccc6c6ccccc65)c4)cc3)nc(-n3c4ccccc4c4ccccc43)n2)cc1 ; c1ccc(-c2nc(-c3ccc(-n4c5ccccc5c5ccccc54)cc3)nc(-c3cccc(-n4c5ccccc5c5ccccc54)c3)n2)cc1]

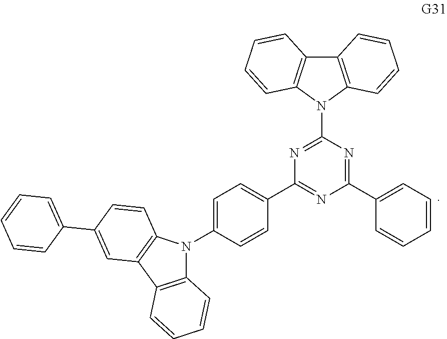 [SMILES: c1ccc(-c2ccc3c(c2)c2ccccc2n3-c2ccc(-c3nc(-c4ccccc4)nc(-n4c5ccccc5c5ccccc54)n3)cc2)cc1]

According to one embodiment, the composition comprises b) at least one organic metal complex, wherein the metal of the organic metal complex is selected from alkali or alkaline earth metal, more preferably the metal is lithium, magnesium or calcium, further preferred the metal is lithium.

According to one embodiment, the composition comprises one organic metal complex.

According to one embodiment, the composition consists of a compound of formula (1) and an organic metal complex, wherein the metal of the organic metal complex is selected from alkali, alkaline earth or rare earth metal, more preferably the metal is an alkali or alkaline earth metal, further preferred the metal is lithium.

According to one embodiment, the composition comprises b) at least one organic metal complex, wherein the organic metal complex comprises at least one ligand, wherein the ligand is selected from a quinolate or borate group, preferably a quinolate group.

Quinolates that can be suitable used are disclosed in WO 2013079217 A1 and incorporated by reference.

Borate groups that can be suitable used are disclosed in WO 2013079676 A1.

According to one embodiment, the composition comprises at least one organic metal complex, wherein the organic metal complex the following Formula 5

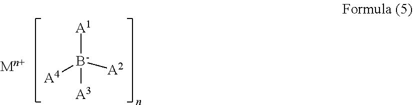

wherein M is a metal ion, each of A¹-A⁴ is independently selected from H, substituted or unsubstituted C₆ to C₂₀ aryl and substituted or unsubstituted C₂ to C₂₀ heteroaryl and n is valency of the metal ion.

According to one embodiment of formula (5), wherein n is 1 or 2.

According to one embodiment of formula (5), wherein M is an alkali metal, an alkaline earth metal or a rare earth metal, alternatively an alkali metal or alkaline earth metal, alternatively selected from lithium, magnesium or calcium.

According to one embodiment of formula (5), wherein at least three groups selected from A¹ to A⁴ are nitrogen containing heteroaryl.

According to one embodiment, wherein the heteroaryl of Formula (5) contains a nitrogen and the nitrogen containing heteroaryl is bound to the central boron atom via a N—N bond, preferably the heteroaryl in Formula (5) is pyrazolyl.

The compound of formula (1) and/or the organic metal complex may be essentially non-emissive.

According to one embodiment, the composition of the present invention may be used in an electron transport layer. Preferably the composition of the present invention comprising the compound of formula 1 and at least one organic metal complex, wherein the metal of the organic metal complex is selected from the group comprising alkali, alkaline earth or rare earth metal, may be used in an electron transport layer.

According to another aspect an organic semiconductor layer may comprises at least one composition of the present invention.

The organic semiconductor layer comprising the composition of the present invention may be essentially non-emissive.

The thickness of the organic semiconductor layer may be from about 0.5 nm to about 100 nm, for example about 2 nm to about 40 nm. When the thickness of the organic semiconductor layer is within these ranges, the organic semiconductor layer may have improved charge transport ability without a substantial increase in operating voltage.

The organic semiconductor layer comprising the composition of the present invention may have strong electron transport characteristics to increase charge mobility and/or stability.

According to one embodiment of the present invention, the organic semiconductor layer is an electron transport layer.

According to another aspect an electronic device may comprises at least one organic semiconductor layer of the present invention.

According to another aspect an electronic device may comprises at least one anode and at least one cathode, preferably the organic semiconductor layer is arranged between the anode and the cathode.

The organic semiconductor layer comprising a composition of the present invention may have strong electron transport characteristics to increase charge mobility and/or stability and thereby to improve luminance efficiency, voltage characteristics, and/or lifetime characteristics of an electronic device.

The electronic device of the present invention may further comprise a photoactive layer, wherein the organic semiconductor layer of the present invention is arranged between the photoactive layer and the cathode layer, preferably between an emission layer or light-absorbing layer and the cathode layer, preferably the organic semiconductor layer is an electron transport layer.

An organic electronic device according to one embodiment comprises the organic semiconductor layer of the present invention, at least one anode layer, at least one cathode layer and at least one emission layer, wherein the organic semiconductor layer is preferably arranged between the emission layer and the cathode layer.

An organic electronic device according to one embodiment can be a light emitting device, thin film transistor, a battery, a display device or a photovoltaic cell, and preferably a light emitting device. A light emitting device can be an OLED.

According to one embodiment the OLED may have the following layer structure, wherein the layers having the following order:

an anode layer, a hole injection layer, optional a first hole transport layer, optional a second hole transport layer, an emission layer, an electron transport layer comprising the composition according to the invention, an electron injection layer, and a cathode layer.

According to another aspect of the present invention, there is provided a method of manufacturing an organic electronic device, the method using:
at least one deposition source, preferably two deposition sources and more preferred at least three deposition sources.

The methods for deposition that can be suitable comprise:
deposition via vacuum thermal evaporation;
deposition via solution processing, preferably the processing is selected from spin-coating, printing, casting; and/or
slot-die coating.

According to various embodiments of the present invention, there is provided a method using:
a first deposition source to release the compound of formula 1 according to the invention, and
a second deposition source to release the at least one organic metal complex; the method comprising the steps of forming the electron transport layer stack; whereby for an organic light-emitting diode (OLED):
the first electron transport layer is formed by releasing the compound of formula 1 from the first deposition source and the organic metal complex from the second deposition source.

According to various embodiments of the present invention, the method may further include forming on the anode electrode an emission layer and at least one layer selected from the group consisting of forming a hole injection layer, forming a hole transport layer, or forming a hole blocking layer, between the anode electrode and the organic semiconductor layer.

According to various embodiments of the present invention, the method may further include the steps for forming an organic light-emitting diode (OLED), wherein
on a substrate a first anode electrode is formed,
on the first anode electrode an emission layer is formed,
on the emission layer an electron transport layer stack is formed, preferably a first electron transport layer is formed on the emission layer and a second electron transport layer is formed on the first electron transport layer and the second electron transport layer comprises the composition according to the invention,
and finally a cathode electrode is formed,
optional a hole injection layer, a hole transport layer, and a hole blocking layer, formed in that order between the first anode electrode and the emission layer,
optional an electron injection layer is formed between the electron transport layer stack and the cathode electrode.

According to various embodiments of the present invention, the method may further include forming an electron injection layer on a first electron transport layer. However, according to various embodiments of the OLED of the present invention, the OLED may not comprise an electron injection layer.

According to another aspect of the invention, it is provided an electronic device comprising at least one organic light emitting device according to any embodiment described throughout this application, preferably, the electronic device comprises the organic light emitting diode in one of embodiments described throughout this application. More preferably, the electronic device is a display device.

The term “organic metal complex” means a compound which comprises one or more metal and one or more organic groups. The metal may be bound to the organic group via a covalent or ionic bond. The organic group means a group comprising mainly covalently bound carbon and hydrogen atoms. The organic group may further comprise heteroatoms selected from N, O, S, B, Si, P, Se, preferably from B, N, O and S.

In the context of the present specification the term “essentially non-emissive” or “non-emitting” means that the visible emission spectrum from the composition or a layer of a) the compound of formula 1 and b) at least one organic metal complex, wherein the metal of the organic metal complex is selected from the group comprising alkali, alkaline earth or rare earth metal in a device is less than 10%, preferably less than 5%, further preferred less than 1%, relative to the visible emission spectrum. The visible emission spectrum is an emission spectrum with a wavelength of about ≥380 nm to about ≤780 nm. Preferably, an organic semiconductor layer or a device comprising a layer, which comprises a) the compound of formula 1 and b) at least one organic metal complex, wherein the metal of the organic metal complex is selected from the group comprising alkali, alkaline earth or rare earth metal, is essentially non-emissive or non-emitting.

The term “free of”, “does not contain”, “does not comprise” does not exclude impurities which may be present in the compounds prior to deposition. Impurities have no technical effect with respect to the object achieved by the present invention.

The operating voltage, also named U, is measured in Volt (V) at 10 milliAmpere per square centimeter (mA/cm2).

The candela per Ampere efficiency, also named cd/A efficiency, is measured in candela per ampere at 10 milli Ampere per square centimeter (mA/cm2).

The external quantum efficiency, also named EQE, is measured in percent (%).

The color space is described by coordinates CIE-x and CIE-y (International Commission on Illumination 1931). For blue emission the CIE-y is of particular importance. A smaller CIE-y denotes a deeper blue color.

The highest occupied molecular orbital, also named HOMO, and lowest unoccupied molecular orbital, also named LUMO, are measured in electron volt (eV).

The rate onset temperature is measured in ° C. and describes the VTE source temperature at which measurable evaporation of a compound commences at a pressure of less than 10′⁵ mbar.

The term “OLED”, “organic light emitting diode”, “organic light emitting device”, “organic optoelectronic device” and “organic light-emitting diode” are simultaneously used and have the same meaning.

The term “transition metal” means and comprises any element in the d-block of the periodic table, which comprises groups 3 to 12 elements on the periodic table.

The term “group III to VI metal” means and comprises any metal in groups III to VI of the periodic table.

As used herein, “weight percent”, “wt.-%”, “percent by weight”, “% by weight”, and variations thereof refer to a composition, component, substance or agent as the weight of that composition, component, substance or agent of the respective electron transport layer divided by the total weight of the composition thereof and multiplied by 100. It is understood that the total weight percent amount of all components, substances or agents of the respective electron transport layer are selected such that it does not exceed 100 wt.-%.

As used herein, “volume percent”, “vol.-%”, “percent by volume”, “% by volume”, and variations thereof refer to an elemental metal, a composition, component, substance or agent as the volume of that elemental metal, component, substance or agent of the respective electron transport layer divided by the total volume of the respective electron transport layer thereof and multiplied by 100. It is understood that the total volume percent amount of all elemental metal, components, substances or agents of the respective cathode electrode layer are selected such that it does not exceed 100 vol.-%.

All numeric values are herein assumed to be modified by the term “about”, whether or not explicitly indicated. As used herein, the term “about” refers to variation in the numerical quantity that can occur.

Whether or not modified by the term “about”, the claims include equivalents to the quantities.

It should be noted that, as used in this specification and the appended claims, the singular forms “a”, “an”, and “the” include plural referents unless the content clearly dictates otherwise.

It should be noted that, as used in this specification and the appended claims, “*” if not otherwise defined indicates the chemical bonding position.

The anode electrode and cathode electrode may be described as anode electrode/cathode electrode or anode electrode/cathode electrode or anode electrode layer/cathode electrode layer.

In the present specification, when a definition is not otherwise provided, an “alkyl group” may refer to an aliphatic hydrocarbon group. The alkyl group may refer to “a saturated alkyl group” without any double bond or triple bond. The alkyl group may be a linear, cyclic or branched alkyl group.

The alkyl group may be a C₁ to C₁₆ alkyl group, or preferably a C₁ to C₁₂ alkyl group. More specifically, the alkyl group may be a C₁ to C₁₄ alkyl group, or preferably a C₁ to C₁₀ alkyl group or a C₁ to C₆ alkyl group. For example, a C₁ to C₄ alkyl group comprises 1 to 4 carbons in alkyl chain, and may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a tert-butyl group, a pentyl group, a hexyl group, a cyclopropyl group, a cyclobutyl group, a cyclopentyl group, a cyclohexyl group, and the like.

In the present specification, “aryl” and “arylene group” may refer to a group comprising at least one hydrocarbon aromatic moiety, and all the elements of the hydrocarbon aromatic moiety may have p-orbitals which form conjugation, for example a phenyl group, a naphthyl group, an anthracenyl group, a phenanthrenyl group, a pyrenyl group, a fluorenyl group and the like.

The term “heteroaryl” and “heteroarylene” may refer to aromatic heterocycles with at least one heteroatom, and all the elements of the aromatic heterocycle may have p-orbitals which form conjugation, for example a pyridyl, pyrimidyl, pyrazinyl, triazinyl, pyrrolyl, carbazolyl, furanyl, benzofuranyl, dibenzofuranyl, thiophenyl, benzothiophenyl, dibenzothiophenyl group and the like. Preferably, the aromatic heterocycles are free of sp³-hybridised carbon atoms.

The term “substituted or unsubstituted heteroaryl”, “substituted or unsubstituted C₅ to C₂₄ heteroaryl”, “substituted or unsubstituted C₃ to C₂₄ heteroaryl”, “substituted or unsubstituted C₅ to C₁₈ heteroaryl”, “substituted or unsubstituted C₅ to C₁₇ heteroaryl”, “substituted or unsubstituted C₃ to C₁₇ heteroaryl”, “substituted or unsubstituted C₃ to C₁₂ heteroarylene” and the like means that the substituted or unsubstituted heteroaryl comprises at least one heteroaryl ring; or at least one heteroaryl ring and at least one non-heteroaryl ring; or at least two heteroaryl rings and at least one non-heteroaryl ring; or at least three heteroaryl rings and at least one non-heteroaryl ring; or at least one heteroaryl ring and at least two non-heteroaryl rings. The rings of the substituted or unsubstituted heteroaryl can be a fused.

The term “hetero-fluorene ring” refers to a dibenzo[d,d]furanyl, dibenzo[b,d]thiophenyl or dibenzo[b,d]selenophenyl group.

The heteroatom may be selected from N, O, S, B, Si, P, Se, preferably from N, O and S.

A heteroarylene ring may comprise at least 1 to 3 heteroatoms. Preferably a heteroarylene ring may comprise at least 1 to 3 heteroatoms individually selected from N, S and/or O.

Further preferred in addition to the compounds of formula 1 at least one additional heteroaryl/ene ring may comprise at least 1 to 3 N-atoms, or at least 1 to 2-N atoms or at least one N-atom.

Further preferred in addition to the compounds of formula 1 at least one additional heteroaryl/ene ring may comprise at least 1 to 3 O-atoms, or at least 1 to 2 O-atoms or at least one O-atom.

Further preferred in addition to the compounds of formula 1 at least one additional heteroaryl/ene ring may comprise at least 1 to 3 S-atoms, or at least 1 to 2 S-atoms or at least one S-atom.

According to another preferred embodiment the compound according to formula 1 may comprise:
at least 6 to 25 aromatic rings, preferably at least 7 to 22 aromatic rings, further preferred at least 8 to 20 aromatic rings, in addition preferred at least 9 to 15 aromatic rings and more preferred at least 10 to 14 aromatic rings; wherein
at least 2 to 5, preferably 3 to 4 or 2 to 3 are heteroaromatic rings.

According to one embodiment the compound according to formula 1:
comprises at least about 6 to about 20 aromatic rings, preferably at least about 7 to about 18 aromatic rings, further preferred at least about 9 to about 16 aromatic rings, in addition preferred at least about 10 to about 15 aromatic rings and more preferred at least about 11 to about 14 aromatic rings; and/or
the compound of formula 1 comprises at least about 2 to about 6, preferably about 3 to about 5 or about 2 to about 4, hetero aromatic rings, wherein the hetero atoms can be selected from N, O, S.

According to one embodiment the compound according to formula 1 can be free of a fluorene ring and free of a hetero-fluorene ring.

According to one embodiment the compound according to formula 1 can be free of a spiro-group.

According to a further preferred embodiment the compound of formula 1 comprises at least 2 to 7, preferably 2 to 5, or 2 to 3 hetero aromatic rings.

According to a further preferred embodiment the compound of formula 1 comprises at least 2 to 7, preferably 2 to 5, or 2 to 3 hetero aromatic rings, wherein at least one of the aromatic rings is a five member hetero aromatic ring.

According to a further preferred embodiment the compound of formula 1 comprises at least 3 to 7, preferably 3 to 6, or 3 to 5 hetero aromatic rings, wherein at least two of the hetero aromatic rings are five member hetero-aromatic-rings.

According to one embodiment the compound according to formula 1 may comprise at least 6 to 12 non-hetero aromatic rings and 2 to 3 hetero aromatic rings.

According to one preferred embodiment the compound according to formula 1 may comprise at least 7 to 12 non-hetero aromatic rings and 2 to 5 hetero aromatic rings.

According to one preferred embodiment the compound according to formula 1 may comprise at least 7 to 11 non-hetero aromatic rings and 2 to 3 hetero aromatic rings.

### Melting Point

The melting point (mp) is determined as peak temperatures from the DSC curves of the above TGA-DSC measurement or from separate DSC measurements (Mettler Toledo DSC822e, heating of samples from room temperature to completeness of melting with heating rate 10 K/min under a stream of pure nitrogen. Sample amounts of 4 to 6 mg are placed in a 40 μL Mettler Toledo aluminum pan with lid, a <1 mm hole is pierced into the lid).

According to another embodiment the compound of formula 1 may have a melting point of about ≥250° C. and about ≤380° C., preferably about ≥260° C. and about ≤370° C., further preferred about ≥265° C. and about ≤360° C.

### Glass Transition Temperature

The glass transition temperature is measured under nitrogen and using a heating rate of 10 K per min in a Mettler Toledo DSC 822e differential scanning calorimeter as described in DIN EN ISO 11357, published in March 2010.

According to another embodiment the compound of formula 1 may have a glass transition temperature Tg of about ≥105° C. and about ≤380° C., preferably about ≥110° C. and about ≤350° C.

### Rate Onset Temperature

The rate onset temperature is determined by loading 100 mg compound into a VTE source. As VTE source a point source for organic materials is used as supplied by Kurt J. Lesker Company (www.lesker.com) or CreaPhys GmbH (http://www.creaphys.com). The VTE source is heated at a constant rate of 15 K/min at a pressure of less than 10′⁵ mbar and the temperature inside the source measured with a thermocouple. Evaporation of the compound is detected with a QCM detector which detects deposition of the compound on the quartz crystal of the detector. The deposition rate on the quartz crystal is measured in Angstrom per second. To determine the rate onset temperature, the deposition rate is plotted against the VTE source temperature. The rate onset is the temperature at which noticeable deposition on the QCM detector occurs. For accurate results, the VTE source is heated and cooled three time and only results from the second and third run are used to determine the rate onset temperature.

To achieve good control over the evaporation rate of an organic compound, the rate onset temperature may be in the range of 200 to 255° C. If the rate onset temperature is below 200° C. the evaporation may be too rapid and therefore difficult to control. If the rate onset temperature is above 255° C. the evaporation rate may be too low which may result in low takt time and decomposition of the organic compound in VTE source may occur due to prolonged exposure to elevated temperatures.

The rate onset temperature is an indirect measure of the volatility of a compound. The higher the rate onset temperature the lower is the volatility of a compound.

According to another embodiment the compound of formula 1 may have a rate onset temperature TRO of about ≥200° C. and about ≤260° C., preferably about ≥220° C. and about ≤260° C., further preferred about ≥220° C. and about ≤260° C., in addition preferred about ≥230° C. and about ≤255° C.

### Dipole Moment

The dipole moment |\vec{μ}| of a molecule containing N atoms is given by:

$$\vec{μ} = {\sum }_{i}^{N} {q}_{i} {\vec{r}}_{i}$$

$$\vert \vec{μ} \vert = \sqrt{{μ}_{x}^{2} + {μ}_{y}^{2} + {μ}_{z}^{2}}$$

where qᵢ and \vec{r}ᵢ are the partial charge and position of atom i in the molecule.

The dipole moment is determined by a semi-empirical molecular orbital method. The geometries of the molecular structures are optimized using the hybrid functional B3LYP with the 6-31G* basis set in the gas phase as implemented in the program package TURBOMOLE V6.5 (TURBOMOLE GmbH, Litzenhardtstrasse 19, 76135 Karlsruhe, Germany). If more than one conformation is viable, the conformation with the lowest total energy is selected to determine the bond lengths of the molecules.

According to one embodiment the compounds according to formula 1 may have a dipole moment (Debye) in the range from about ≥1.2 to about ≤4, preferably from about ≥1.3 to about ≤3.8, further preferred from about ≥1.4 to about ≤3.6.

### Calculated HOMO and LUMP

The HOMO and LUMO are calculated with the program package TURBOMOLE V6.5. The optimized geometries and the HOMO and LUMO energy levels of the molecular structures are determined by applying the hybrid functional B3LYP with a 6-31G* basis set in the gas phase. If more than one conformation is viable, the conformation with the lowest total energy is selected.

According to one embodiment the compounds according to formula 1 may have a LUMO energy level (eV) in the range from about −2.20 eV to about −1.90 eV, preferably from about −2.1 eV to about −1.91 eV, further preferred from about −2.08 eV to about −1.92 eV, also preferred from about −2.06 eV to about −1.95 eV.

### Technical Effect

Surprisingly, it was found that the composition according to invention and the inventive organic electronic devices solve the problem underlying the present invention by being superior over the organic electroluminescent devices and compositions known in the art, in particular with respect to lifetime. At the same time the operating voltage is kept at a similar or even improved level which is important for reducing power consumption and increasing battery life, for example of a mobile display device. Long lifetime at high current density is important for the longevity of a device which is run at high brightness.

Additionally, it was surprisingly found that the calculated LUMO level of compounds of formula 1 is significantly more negative than the LUMO of the state of the art. A more negative LUMO may be beneficial for improved electron transfer from the cathode to the emission layer.

Furthermore, it was surprisingly found that the rate onset temperature of compounds of formula 1 is significantly lower than of the state of the art. A lower rate onset temperature may be beneficial for mass production as the deposition rate can be increased without increasing decomposition of the compound in the VTE source.

The inventors have surprisingly found that particular good performance can be achieved when using the organic electroluminescent device as a fluorescent blue device.

The specific arrangements mentioned herein as preferred were found to be particularly advantageous.

Likewise, some compounds falling within the scope of the broadest definition of the present invention have surprisingly be found to be particularly well performing with respect to the mentioned property of cd/A efficiency and/or lifetime. These compounds are discussed herein to be particularly preferred.

Further an organic optoelectronic device having high efficiency and/or long lifetime may be realized.

### Anode

A material for the anode may be a metal or a metal oxide, or an organic material, preferably a material with work function above about 4.8 eV, more preferably above about 5.1 eV, most preferably above about 5.3 eV. Preferred metals are noble metals like Pt, Au or Ag, preferred metal oxides are transparent metal oxides like ITO or IZO which may be advantageously used in bottom-emitting OLEDs having a reflective cathode.

In devices comprising a transparent metal oxide anode or a reflective metal anode, the anode may have a thickness from about 50 nm to about 100 nm, whereas semitransparent metal anodes may be as thin as from about 5 nm to about 15 nm, and non-transparent metal anodes may have a thickness from about 15 nm to about 150 nm.

### Hole Injection Layer (HIL)

The hole injection layer may improve interface properties between the anode and an organic material used for the hole transport layer, and is applied on a non-planarized anode and thus may planarize the surface of the anode. For example, the hole injection layer may include a material having a median value of the energy level of its highest occupied molecular orbital (HOMO) between the work function of the anode material and the energy level of the HOMO of the hole transport layer, in order to adjust a difference between the work function of the anode and the energy level of the HOMO of the hole transport layer.

When the hole transport region comprises a hole injection layer 36, the hole injection layer may be formed on the anode by any of a variety of methods, for example, vacuum deposition, spin coating, casting, Langmuir-Blodgett (LB) method, or the like.

When hole injection layer is formed using vacuum deposition, vacuum deposition conditions may vary depending on the material that is used to form the hole injection layer, and the desired structure and thermal properties of the hole injection layer to be formed and for example, vacuum deposition may be performed at a temperature of about 100° C. to about 500° C., a pressure of about 10⁻⁶ Pa to about 10⁻¹ Pa, and a deposition rate of about 0.1 to about 10 nm/sec, but the deposition conditions are not limited thereto.

When the hole injection layer is formed using spin coating, the coating conditions may vary depending on the material that is used to form the hole injection layer, and the desired structure and thermal properties of the hole injection layer to be formed. For example, the coating rate may be in the range of about 2000 rpm to about 5000 rpm, and a temperature at which heat treatment is performed to remove a solvent after coating may be in a range of about 80° C. to about 200° C., but the coating conditions are not limited thereto.

The hole injection layer may further comprise a p-dopant to improve conductivity and/or hole injection from the anode.

p-Dopant

In another aspect, the p-dopant may be homogeneously dispersed in the hole injection layer.

In another aspect, the p-dopant may be present in the hole injection layer in a higher concentration closer to the anode and in a lower concentration closer to the cathode.

The p-dopant may be one of a quinone derivative or a radialene compound but not limited thereto. Non-limiting examples of the p-dopant are quinone derivatives such as tetracyanoquinonedimethane (TCNQ), 2,3,5,6-tetrafluoro-tetracyano-1,4-benzoquinonedimethane (F4-TCNQ), 4,4′,4″-((1E,1′E, 1″E)-cyclopropane-1,2,3-triylidenetris(cyanomethanylylidene))-tris(2,3,5,6-tetrafluorobenzonitrile).

According to another embodiment, an organic electronic device comprising an organic semiconductor layer comprising a composition according to invention may additional comprise a layer comprising a radialene compound and/or a quinodimethane compound.

In another embodiment, the radialene compound and/or the quinodimethane compound may be substituted with one or more halogen atoms and/or with one or more electron withdrawing groups. Electron withdrawing groups can be selected from nitrile groups, halogenated alkyl groups, alternatively from perhalogenated alkyl groups, alternatively from perfluorinated alkyl groups. Other examples of electron withdrawing groups may be acyl, sulfonyl groups or phosphoryl groups.

Alternatively, acyl groups, sulfonyl groups and/or phosphoryl groups may comprise halogenated and/or perhalogenated hydrocarbyl. In one embodiment, the perhalogenated hydrocarbyl may be a perfluorinated hydrocarbyl. Examples of a perfluorinated hydrocarbyl can be perfluormethyl, perfluorethyl, perfluorpropyl, perfluorisopropyl, perfluorobutyl, perfluorophenyl, perfluorotolyl; examples of sulfonyl groups comprising a halogenated hydrocarbyl may be trifluoromethylsulfonyl, pentafluoroethylsulfonyl, pentafluorophenylsulfonyl, heptafluoropropylsufonyl, nonafluorobutylsulfonyl, and like.

In one embodiment, the radialene and/or the quinodimethane compound may be comprised in a hole injection, hole transporting and/or a hole generation layer.

In one embodiment, the radialene compound may have formula (XX) and/or the quinodimethane compound may have formula (XXIa) or (XXIb):

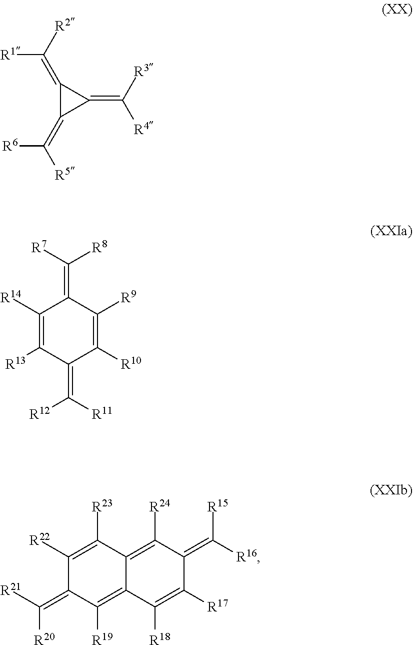

wherein R^{1″}, R^{2″}, R^{3″}, R^{4″}, R^{5″}, R⁶, R⁷, R⁸, R¹¹, R¹², R¹⁵, R¹⁶, R²⁰, R²¹ are independently selected from an electron withdrawing groups and R⁹, R¹⁰, R¹³, R¹⁴, R¹⁷, R¹⁸, R¹⁹, R²², R²³ and R²⁴ are independently selected from H, halogen and electron withdrawing groups. Electron withdrawing group/s that can be suitable used are above mentioned.

### Hole Transport Layer (HTL)

Conditions for forming the hole transport layer and the electron blocking layer may be defined based on the above-described formation conditions for the hole injection layer.

A thickness of the hole transport part of the charge transport region may be from about 10 nm to about 1000 nm, for example, about 10 nm to about 100 nm. When the hole transport part of the charge transport region comprises the hole injection layer and the hole transport layer, a thickness of the hole injection layer may be from about 10 nm to about 1000 nm, for example about 10 nm to about 100 nm and a thickness of the hole transport layer may be from about 5 nm to about 200 nm, for example about 10 nm to about 150 nm. When the thicknesses of the hole transport part of the charge transport region, the HIL, and the HTL are within these ranges, satisfactory hole transport characteristics may be obtained without a substantial increase in operating voltage.

Hole transport matrix materials used in the hole transport region are not particularly limited. Preferred are covalent compounds comprising a conjugated system of at least 6 delocalized electrons, preferably organic compounds comprising at least one aromatic ring, more preferably organic compounds comprising at least two aromatic rings, even more preferably organic compounds comprising at least three aromatic rings, most preferably organic compounds comprising at least four aromatic rings. Typical examples of hole transport matrix materials which are widely used in hole transport layers are polycyclic aromatic hydrocarbons, triarylene amine compounds and heterocyclic aromatic compounds. Suitable ranges of frontier orbital energy levels of hole transport matrices useful in various layer of the hole transport region are well-known. In terms of the redox potential of the redox couple HTL matrix/cation radical of the HTL matrix, the preferred values (if measured for example by cyclic voltammetry against ferrocene/ferrocenium redox couple as reference) may be in the range 0.0-1.0 V, more preferably in the range 0.2-0.7 V, even more preferably in the range 0.3-0.5 V.

### Buffer Layer

The hole transport part of the charge transport region may further include a buffer layer.

Buffer layer that can be suitable used are disclosed in U.S. Pat. Nos. 6,140,763, 6,614,176 and in US2016/248022.

The buffer layer may compensate for an optical resonance distance of light according to a wavelength of the light emitted from the EML, and thus may increase efficiency.

### Emission Layer (EMU)

The emission layer may be formed on the hole transport region by using vacuum deposition, spin coating, casting, LB method, or the like. When the emission layer is formed using vacuum deposition or spin coating, the conditions for deposition and coating may be similar to those for the formation of the hole injection layer, though the conditions for the deposition and coating may vary depending on the material that is used to form the emission layer. The emission layer may include an emitter host (EML host) and an emitter dopant (further only emitter).

A thickness of the emission layer may be about 100 Å to about 1000 Å, for example about 200 Å to about 600 Å. When the thickness of the emission layer is within these ranges, the emission layer may have improved emission characteristics without a substantial increase in operating voltage.

### Emitter Host

According to another embodiment, the emission layer comprises compound of formula 1 as emitter host.

The emitter host compound has at least three aromatic rings, which are independently selected from carbocyclic rings and heterocyclic rings.

Other compounds that can be used as the emitter host is an anthracene matrix compound represented by formula 400 below:

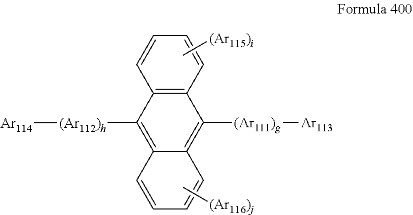

In formula 400, Ar₁₁₁ and Ar₁₁₂ may be each independently a substituted or unsubstituted C₆-C₆₀ arylene group; Ar₁₁₃ to Ar₁₁₆ may be each independently a substituted or unsubstituted C₁-C₁₀ alkyl group or a substituted or unsubstituted C₆-C₆₀ arylene group; and g, h, i, and j may be each independently an integer from 0 to 4.

In some embodiments, Ar₁₁₁ and Ar₁₁₂ in formula 400 may be each independently one of a phenylene group, a naphthalene group, a phenanthrenylene group, or a pyrenylene group; or

a phenylene group, a naphthalene group, a phenanthrenylene group, a fluorenyl group, or a pyrenylene group, each substituted with at least one of a phenyl group, a naphthyl group, or an anthryl group.

In formula 400, g, h, i, and j may be each independently an integer of 0, 1, or 2.

In formula 400, Ar₁₁₃ to Ar₁₁₆ may be each independently one of
a C₁-C₁₀ alkyl group substituted with at least one of a phenyl group, a naphthyl group, or an anthryl group;
a phenyl group, a naphthyl group, an anthryl group, a pyrenyl group, a phenanthrenyl group, or a fluorenyl group;
a phenyl group, a naphthyl group, an anthryl group, a pyrenyl group, a phenanthrenyl group, or a fluorenyl group, each substituted with at least one of a deuterium atom, a halogen atom, a hydroxyl group, a cyano group, a nitro group, an amino group, an amidino group, a hydrazine group, a hydrazone group, a carboxyl group or a salt thereof,
a sulfonic acid group or a salt thereof, a phosphoric acid group or a salt thereof,
a C₁-C₆₀ alkyl group, a C₂-C₆₀ alkenyl group, a C₂-C₆₀ alkynyl group, a C₁-C₆₀ alkoxy group, a phenyl group, a naphthyl group, an anthryl group, a pyrenyl group, a phenanthrenyl group, or
a fluorenyl group

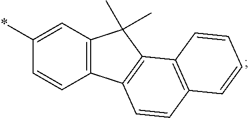 [SMILES: *c1ccc2c(c1)C(C)(C)c1c-2ccc2ccccc12]

or
formulas 7 or 8

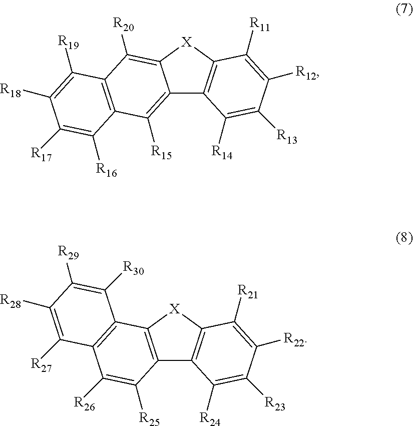

Wherein in the formulas 7 and 8, X is selected form an oxygen atom and a sulfur atom, but embodiments of the invention are not limited thereto.

In the formula 7, any one of R₁₁ to R₁₄ is used for bonding to Ar₁₁₁. R₁₁ to R₁₄ that are not used for bonding to Arm and R₁₅ to R₂₀ are the same as R₁ to R₈.

In the formula 8, any one of R₂₁ to R₂₄ is used for bonding to Ar₁₁₁. R₂₁ to R₂₄ that are not used for bonding to Arm and R₂₅ to R₃₀ are the same as R₁ to R₈.

Preferably, the EML host comprises between one and three heteroatoms selected from the group consisting of N, O or S. More preferred the EML host comprises one heteroatom selected from S or O.

### Emitter Dopant

The dopant is mixed in a small amount to cause light emission, and may be generally a material such as a metal complex that emits light by multiple excitation into a triplet or more. The dopant may be, for example an inorganic, organic, or organic/inorganic compound, and one or more kinds thereof may be used.

The emitter may be a red, green, or blue emitter.

The dopant may be a fluorescent dopant, for example ter-fluorene, the structures are shown below. 4.4′-bis(4-diphenyl amiostyryl)biphenyl (DPAVBI, 2,5,8,11-tetra-tert-butyl perylene (TBPe), and Compound 8 below are examples of fluorescent blue dopants.

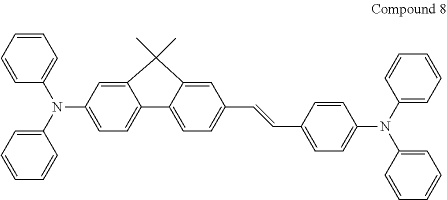 [SMILES: CC1(C)c2cc(/C=C/c3ccc(N(c4ccccc4)c4ccccc4)cc3)ccc2-c2ccc(N(c3ccccc3)c3ccccc3)cc21]

The dopant may be a phosphorescent dopant, and examples of the phosphorescent dopant may be an organic metal compound comprising Ir, Pt, Os, Ti, Zr, Hf, Eu, Tb, Tm, Fe, Co, Ni, Ru, Rh, Pd, or a combination thereof. The phosphorescent dopant may be, for example a compound represented by formula Z, but is not limited thereto:

J₂MX (Z).

In formula Z, M is a metal, and J and X are the same or different, and are a ligand to form a complex compound with M.

The M may be, for example Ir, Pt, Os, Ti, Zr, Hf, Eu, Tb, Tm, Fe, Co, Ni, Ru, Rh, Pd or a combination thereof, and the J and X may be, for example a bidendate ligand.

One or more emission layers may be arranged between the anode and the cathode. To increase overall performance, two or more emission layers may be present.

### Charge Generation Layer

A charge generation layer (also named CGL) may be arranged between the first and the second emission layer, and second and third emission layer, if present. Typically, the CGL comprises a n-type charge generation layer (also named n-CGL or electron generation layer) and a p-type charge generation layer (also named p-CGL or hole generation layer). An interlayer may be arranged between the n-type CGL and the p-type CGL.

In one aspect, the n-type CGL may comprise a matrix compound and a metal, metal salt or organic metal complex, preferably a metal. The metal may be selected from an alkali, alkaline earth or rare earth metal. The organic semiconductor layer comprising the composition according to the invention may be arranged between the first emission layer and the n-CGL and/or between the second and/or third emission layer and the cathode.

The p-type CGL may comprise a dipyrazino[2,3-f:2′,3′-h]quinoxaline, a quinone compound or a radialene compound, preferably dipyrazino[2,3-f:2′,3′-h]quinoxaline-2,3,6,7,10,11-hexacarbonitrile or a compound or formula (XX) and/or a compound of formula (XXIa) or (XXIb).

### Electron Transport Layer (ETL)

According to another embodiment, the organic semiconductor layer that comprises the composition is an electron transport layer. In another embodiment the electron transport layer may consist of the composition according to the invention.

In another embodiment, the organic electronic device comprises an electron transport region of a stack of organic layers formed by two or more electron transport layers, wherein at least one electron transport layer comprises the composition.
The electron transport layer may include one or two or more different compounds of formula (1) and/or organic metal complexes.

The thickness of the electron transport layer may be from about 0.5 nm to about 100 nm, for example about 2 nm to about 40 nm. When the thickness of the electron transport layer is within these ranges, the electron transport layer may have improved electron transport ability without a substantial increase in operating voltage.

### Electron Injection Layer (EIL)

According to another aspect of the invention, the organic electroluminescent device may further comprise an electron injection layer between the electron transport layer (first-ETL) and the cathode.

The electron injection layer (EIL) may facilitate injection of electrons from the cathode.

According to another aspect of the invention, the electron injection layer comprises:
(i) an electropositive metal selected from alkali metals, alkaline earth metals and rare earth metals in substantially elemental form, preferably selected from Li, Na, K, Rb, Cs, Mg, Ca, Sr, Ba, Eu and Yb, more preferably from Li, Na, Mg, Ca, Sr and Yb, even more preferably from Li and Yb, most preferably Yb; and/or
(ii) an alkali metal complex and/or alkali metal salt, preferably the Li complex and/or salt, more preferably a Li quinolinolate, even more preferably a lithium 8-hydroxyquino-linolate, most preferably the alkali metal salt and/or complex of the second electron transport layer (second-ETL) is identical with the alkali metal salt and/or complex of the injection layer.

The electron injection layer may include at least one selected from LiF, NaCl, CsF, Li₂O, and BaO.

A thickness of the EIL may be from about 0.1 nm to about 10 nm, or about 0.3 nm to about 9 nm. When the thickness of the electron injection layer is within these ranges, the electron injection layer may have satisfactory electron injection ability without a substantial increase in operating voltage.

The electron injection layer may comprise or consist of the composition according to the invention.

### Cathode

A material for the cathode may be a metal, an alloy, or an electrically conductive compound that have a low work function, or a combination thereof. Specific examples of the material for the cathode may be lithium (Li), magnesium (Mg), aluminum (Al), aluminum-lithium (Al—Li), calcium (Ca), magnesium-indium (Mg—In), magnesium-silver (Mg—Ag), silver (Ag) etc. In order to manufacture a top-emission light-emitting device having a reflective anode deposited on a substrate, the cathode may be formed as a light-transmissive electrode from, for example, indium tin oxide (ITO), indium zinc oxide (IZO) or silver (Ag).

In devices comprising a transparent metal oxide cathode or a reflective metal cathode, the cathode may have a thickness from about 50 nm to about 100 nm, whereas semitransparent metal cathodes may be as thin as from about 5 nm to about 15 nm.

Substrate

A substrate may be further disposed under the anode or on the cathode. The substrate may be a substrate that is used in a general organic light emitting diode and may be a glass substrate or a transparent plastic substrate with strong mechanical strength, thermal stability, transparency, surface smoothness, ease of handling, and water resistance.

Hereinafter, the embodiments are illustrated in more detail with reference to examples. However, the present disclosure is not limited to the following examples.

### DESCRIPTION OF THE DRAWINGS

These and/or other aspects and advantages of the present invention will become apparent and more readily appreciated from the following description of the exemplary embodiments, taken in conjunction with the accompanying drawings, of which:

FIG. 1 is a schematic sectional view of an organic light-emitting diode (OLED), according to an exemplary embodiment of the present invention with an emission layer, one electron transport layer and an electron injection layer;

FIG. 2 is a schematic sectional view of an organic light-emitting diode (OLED), according to an exemplary embodiment of the present invention with an emission layer and two electron transport layers;

FIG. 3 is a schematic sectional view of an OLED, according to an exemplary embodiment of the present invention with an emission layer and three electron transport layers;

FIG. 4 is a schematic sectional view of an organic light-emitting diode (OLED), according to an exemplary embodiment of the present invention with an emission layer and one electron transport layer;

FIG. 5 is a schematic sectional view of an organic light-emitting diode (OLED), according to an exemplary embodiment of the present invention with an emission layer and two electron transport layers;

FIG. 6 is a schematic sectional view of an OLED, according to an exemplary embodiment of the present invention with an emission layer and three electron transport layers.

Reference will now be made in detail to the exemplary aspects, examples of which are illustrated in the accompanying drawings, wherein like reference numerals refer to the like elements throughout. The exemplary embodiments are described below, in order to explain the aspects, by referring to the figures.

Herein, when a first element is referred to as being formed or disposed “on” a second element, the first element can be disposed directly on the second element, or one or more other elements may be disposed there between. When a first element is referred to as being formed or disposed “directly on” a second element, no other elements are disposed there between.

The term “contacting sandwiched” refers to an arrangement of three layers whereby the layer in the middle is in direct contact with the two adjacent layers.

The organic light emitting diodes according to an embodiment of the present invention may include a hole transport region; an emission layer; and a first electron transport layer comprising the composition according to the invention.

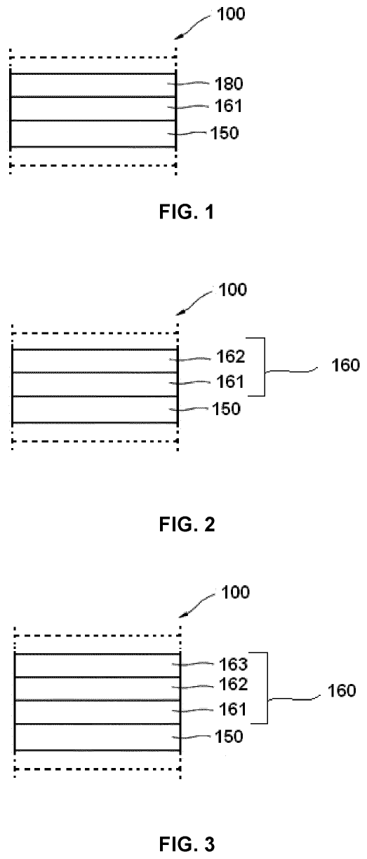

FIG. 1 is a schematic sectional view of an organic light-emitting diode 100, according to an exemplary embodiment of the present invention. The OLED 100 comprises an emission layer 150, an electron transport layer (ETL) 161 comprising the composition according to the invention, and an electron injection layer 180, whereby the first electron transport layer 161 is disposed directly on the emission layer 150 and the electron injection layer 180 is disposed directly on the first electron transport layer 161.

FIG. 2 is a schematic sectional view of an organic light-emitting diode 100, according to an exemplary embodiment of the present invention. The OLED 100 comprises an emission layer 150 and an electron transport layer stack (ETL) 160 comprising a first electron transport layer 161, and a second electron transport layer 162 comprising the composition according to the invention, whereby the second electron transport layer 162 is disposed directly on the first electron transport layer 161.

FIG. 3 is a schematic sectional view of an organic light-emitting diode 100, according to an exemplary embodiment of the present invention. The OLED 100 comprises an emission layer 150 and an electron transport layer stack (ETL) 160 comprising a first electron transport layer 161, a second electron transport layer 162, and a third electron transport layer 163, whereby the second electron transport layer 162 is disposed directly on the first electron transport layer 161 and the third electron transport layer 163 is disposed directly on the first electron transport layer 162. The first and/or the second and/or the third electron transport layer comprise the composition according to the invention.

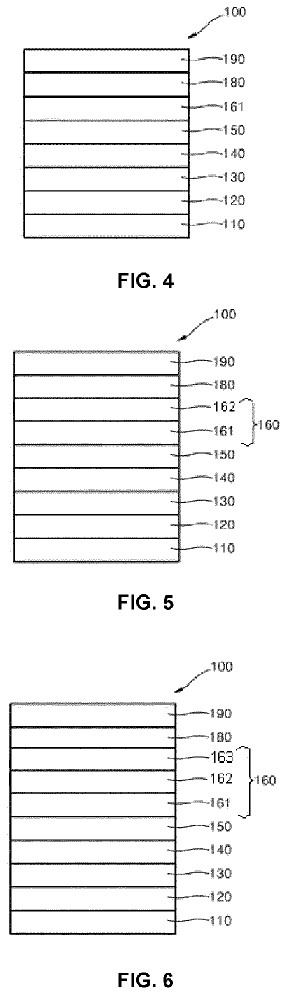

FIG. 4 is a schematic sectional view of an organic light-emitting diode 100, according to an exemplary embodiment of the present invention. The OLED 100 comprises a substrate 110, a first anode electrode 120, a hole injection layer (HIL) 130, a hole transport layer (HTL) 140, an emission layer (EML) 150, one first electron transport layer (ETL) 161, an electron injection layer (EIL) 180, and a cathode electrode 190. The first electron transport layer (ETL) 161 comprises the composition according to the invention. The electron transport layer (ETL) 161 is formed directly on the EML 150.

FIG. 5 is a schematic sectional view of an organic light-emitting diode 100, according to an exemplary embodiment of the present invention. The OLED 100 comprises a substrate 110, a first anode electrode 120, a hole injection layer (HIL) 130, a hole transport layer (HTL) 140, an emission layer (EML) 150, an electron transport layer stack (ETL) 160, an electron injection layer (EIL) 180, and a cathode electrode 190. The electron transport layer (ETL) 160 comprises a first electron transport layer 161 and a second electron transport layer 162, wherein the first electron transport layer is arranged near to the anode (120) and the second electron transport layer is arranged near to the cathode (190). The first and/or the second electron transport layer comprise the composition according to the invention.

FIG. 6 is a schematic sectional view of an organic light-emitting diode 100, according to an exemplary embodiment of the present invention. The OLED 100 comprises a substrate 110, a first anode electrode 120, a hole injection layer (HIL) 130, a hole transport layer (HTL) 140, an emission layer (EML) 150, an electron transport layer stack (ETL) 160, an electron injection layer (EIL) 180, and a second cathode electrode 190. The electron transport layer stack (ETL) 160 comprises a first electron transport layer 161, a second electron transport layer 162 and a third electron transport layer 163. The first electron transport layer 161 is formed directly on the emission layer (EML) 150. The first, second and/or third electron transport layer comprise the composition according to the invention.

Hereinafter, the embodiments are illustrated in more detail with reference to examples. However, the present disclosure is not limited to the following examples. Reference will now be made in detail to the exemplary aspects.

### Preparation of Compounds of Formula 1

Compounds of formula 1 may be prepared as described in US 2015171340. LiQ is commercially available (CAS 25387-93-3). Metal borates may be synthesized as described in WO2013079676A1.

### General Procedure for Fabrication of OLEDs

For top emission devices, Examples 1 to 4 and comparative example 1 and 2, a glass substrate was cut to a size of 50 mm×50 mm×0.7 mm, ultrasonically cleaned with isopropyl alcohol for 5 minutes and then with pure water for 5 minutes, and cleaned again with UV ozone for 30 minutes, to prepare a first electrode. 100 nm Ag were deposited on the glass substrate at a pressure of 10⁻⁵ to 10⁻⁷ mbar to form the anode.

Then, 92 vol.-% Biphenyl-4-yl(9,9-diphenyl-9H-fluoren-2-yl)-[4-(9-phenyl-9H-carbazol-3-yl)phenyl]-amine (CAS 1242056-42-3) with 8 vol.-% 2,2′,2″-(cyclopropane-1,2,3-triylidene)tris(2-(p-cyanotetrafluorophenyl)acetonitrile) was vacuum deposited on the anode, to form a HIE having a thickness of 10 nm.

Then, Biphenyl-4-yl(9,9-diphenyl-9H-fluoren-2-yl)-[4-(9-phenyl-9H-carbazol-3-yl) phenyl]-amine was vacuum deposited on the HIL, to form a HTL having a thickness of 118 nm.

Then N,N-bis(4-(dibenzo[b,d]furan-4-yl)phenyl)-[1,1′:4′,1″-terphenyl]-4-amine (CAS 1198399-61-9) was vacuum deposited on the HTL, to form an electron blocking layer (EBL) having a thickness of 5 nm.

Then 97 vol.-% H09 (Sun Fine Chemicals, South Korea) as EML host and 3 vol.-% BD200 (Sun Fine Chemicals, South Korea) as fluorescent blue dopant were deposited on the EBL, to form a blue-emitting EML with a thickness of 20 nm.

Then the hole blocking layer is formed with a thickness of 5 nm by depositing 2,4-diphenyl-6-(4′,5′,6′-triphenyl-[1,1′:2′,1″:3″,1″:3′″,1″″-quinquephenyl]-3″″-yl)-1,3,5-triazine on the emission layer.

Then, the electron transporting layer is formed on the hole blocking layer according to Examples 1 to 4 and comparative example 1 and 2 with a the thickness of 31 nm. The electron transport layer comprises 50 wt.-% matrix compound and 50 wt.-% of alkali organic complex, see Table 1. In comparative example 2, the electron transport layer comprises 100 wt.-% compound of formula 1 of MX3.

Then, the electron injection layer is formed on the electron transporting layer by deposing Yb with a thickness of 2 nm.

AgMg alloy (90:10 vol.-%) is evaporated at a rate of 0.01 to 1 Å/s at 10⁻⁷ mbar to form a cathode with a thickness of 11 nm.

A cap layer of Biphenyl-4-yl(9,9-diphenyl-9H-fluoren-2-yl)-[4-(9-phenyl-9H-carbazol-3-yl)phenyl]-amine is formed on the cathode with a thickness of 75 nm.

The OLED stack is protected from ambient conditions by encapsulation of the device with a glass slide. Thereby, a cavity is formed, which includes a getter material for further protection.

To assess the performance of the inventive examples compared to the prior art, the current efficiency is measured at 20° C. The current-voltage characteristic is determined using a Keithley 2635 source measure unit, by sourcing a voltage in V and measuring the current in mA flowing through the device under test. The voltage applied to the device is varied in steps of 0.1V in the range between 0V and 10V. Likewise, the luminance-voltage characteristics and CIE coordinates are determined by measuring the luminance in cd/m² using an Instrument Systems CAS-140CT array spectrometer (calibrated by Deutsche Akkreditierungsstelle (DAkkS)) for each of the voltage values. The cd/A efficiency at 10 mA/cm² is determined by interpolating the luminance-voltage and current-voltage characteristics, respectively.

Lifetime LT of the device is measured at ambient conditions (20° C.) and 30 mA/cm², using a Keithley 2400 sourcemeter, and recorded in hours.

The brightness of the device is measured using a calibrated photo diode. The lifetime LT is defined as the time till the brightness of the device is reduced to 97% of its initial value.

### Top Emission Devices

In Table 1 is shown the performance of organic electronic devices comprising an organic semiconductor layer comprising compound of formula 1 and an alkali organic complex.

In comparative example L compound ETM-1 was used as matrix compound:

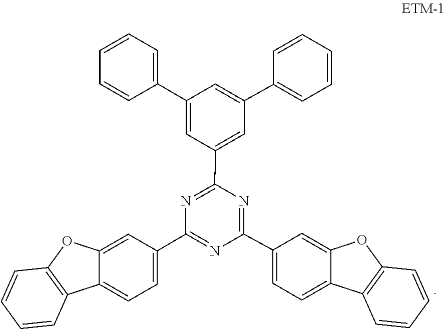 [SMILES: c1ccc(-c2cc(-c3ccccc3)cc(-c3nc(-c4ccc5c(c4)oc4ccccc45)nc(-c4ccc5c(c4)oc4ccccc45)n3)c2)cc1]

Compound ETM-1 is free of carbazole groups. The organic semiconductor layer comprises 50 vol.-% ETM-1 and 50 vol.-% LiQ. The operating voltage is 3.55 V and the cd/A efficiency is 7.3 cd/A. The lifetime LT97 at 30 mA/cm2 is 62 hours.

In comparative example 2, the organic semiconductor layer comprises 100 wt.-% of MX3. The operating voltage is very high at 6 V. The cd/A efficiency is reduced to 5 cd/A. The lifetime LT97 is reduced to 11 hours.

In Example 1, the organic semiconductor layer comprises 50 vol.-% compound of formula 1 of MX1

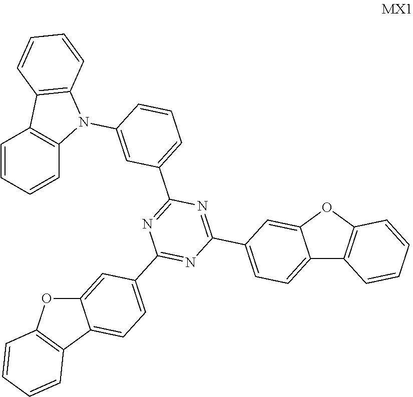 [SMILES: c1cc(-c2nc(-c3ccc4c(c3)oc3ccccc34)nc(-c3ccc4c(c3)oc3ccccc34)n2)cc(-n2c3ccccc3c3ccccc32)c1]

and 50 vol.-% LiQ. The operating voltage is 3.6 V and the cd/A efficiency is 7.0 cd/A. The lifetime is improved to 78 hours.

In Example 2, the organic semiconductor layer comprises 50 vol.-% compound of formula 1 of MX2

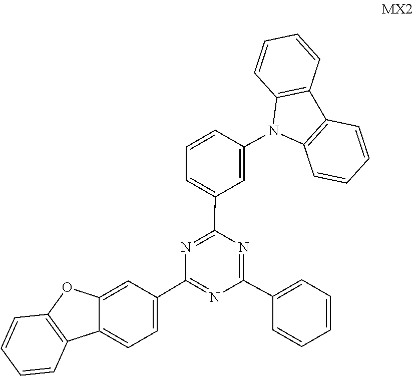 [SMILES: c1ccc(-c2nc(-c3cccc(-n4c5ccccc5c5ccccc54)c3)nc(-c3ccc4c(c3)oc3ccccc34)n2)cc1]

and 50 vol.-% LiQ. The operating voltage is 3.5 V and the cd/A efficiency is 7.0 cd/A. The lifetime is improved to 79 hours.

In Example 3, the organic semiconductor layer comprises 50 vol.-% compound of formula 1 of MX3

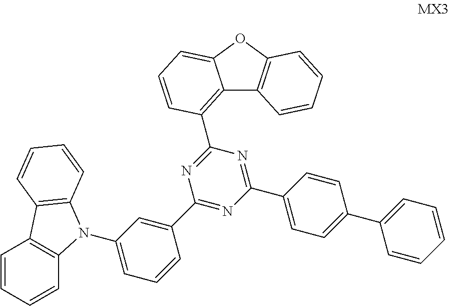 [SMILES: c1ccc(-c2ccc(-c3nc(-c4cccc(-n5c6ccccc6c6ccccc65)c4)nc(-c4cccc5oc6ccccc6c45)n3)cc2)cc1]

and 50 vol.-% LiQ. The operating voltage is 3.6 V and the cd/A efficiency is 7.2 cd/A. The lifetime is improved to 100 hours.

In Example 4, the organic semiconductor layer comprises 50 vol.-% compound of formula 1 of MX4

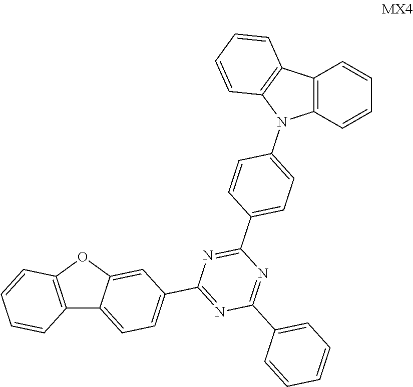 [SMILES: c1ccc(-c2nc(-c3ccc(-n4c5ccccc5c5ccccc54)cc3)nc(-c3ccc4c(c3)oc3ccccc34)n2)cc1]

and 50 vol.-% LiQ. The operating voltage is 3.5 V and the cd/A efficiency is 7.1 cd/A. The lifetime is improved to 91 hours.

TABLE 1
Performance data of organic electroluminescent device comprising an organic semiconductor layer comprising compound of formula 1 and
an alkali organic complex

| | | Concen- | | Concen- | Thick- | Oper- | | |
|---|---|---|---|---|---|---|---|---|
| | | tration | | tration | ness | ating | cd/A | |
| | | of | | of alkali | electron | voltage | effi- | |
| | | matrix | | organic | trans- | at | ciency | LT97 |
| | | com- | Alkali | com- | port | 10 | at 10 | at 30 |
| | | pound | organic | plex | layer | mA/cm² | mA/cm² | mA/cm² |
| | Matrix compound | (vol.-%) | complex | (vol.-%) | (nm) | (V) | (cd/A) | (h) |
| Compar- ative exam- ple 1 | 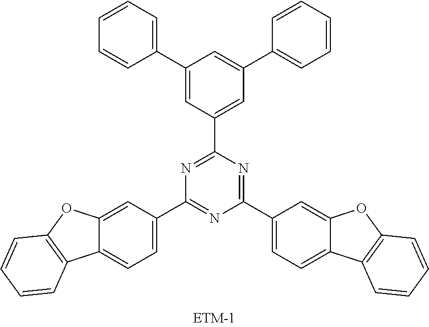 [SMILES: c1ccc(-c2cc(-c3ccccc3)cc(-c3nc(-c4ccc5c(c4)oc4ccccc45)nc(-c4ccc5c(c4)oc4ccccc45)n3)c2)cc1] | 50 | LiQ | 50 | 31 | 3.55 | 7.3 | 62 |
| Compar- ative exam- ple 2 | 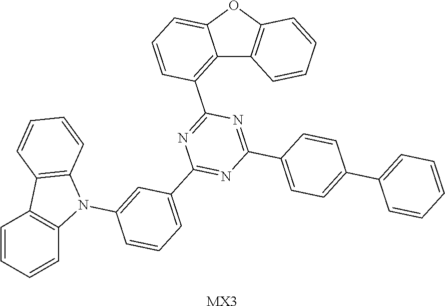 | 100 | — | 0 | 31 | 6 | 6 | 11 |
| Exam- ple 1 | 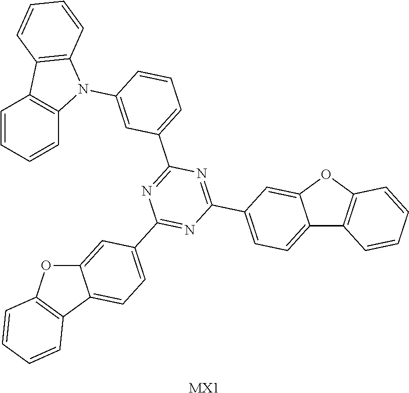 | 50 | LiQ | 50 | 31 | 3.6 | 7.0 | 78 |
| Exam- ple 2 | 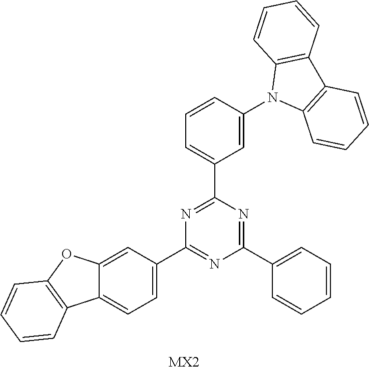 | 50 | LiQ | 50 | 31 | 3.5 | 7.0 | 79 |
| Exam- ple 3 | 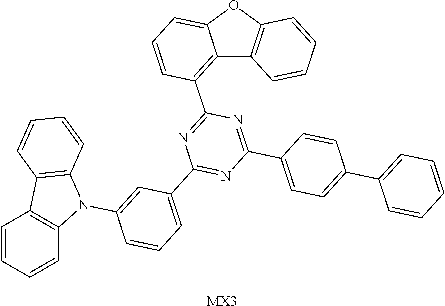 | 50 | LiQ | 50 | 31 | 3.6 | 7.2 | 100 |
| Exam- ple 4 | 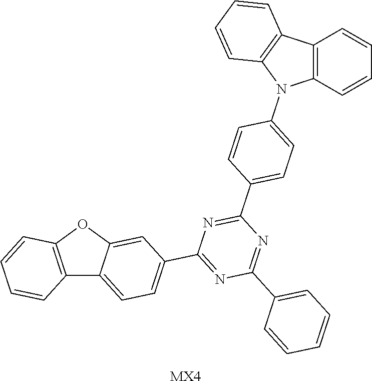 | 50 | LiQ | 50 | 31 | 3.5 | 7.1 | 91 |

In summary, improved lifetime may be achieved when the organic semiconductor layer comprises a compound of formula 1 and an organic metal complex.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

1. A composition comprising
a) a compound of formula 1: 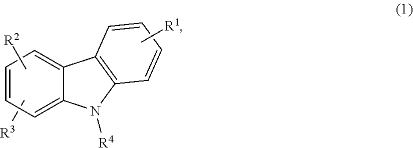
wherein
R¹ and R² are independently selected from H, D, CN, halogen, C₁ to C₁₂ alkyl, C₁ to C₁₂ alkoxy, substituted or unsubstituted C₆ to C₁₈ aryl, substituted or unsubstituted C₃ to C₂₄ heteroaryl,
wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
wherein R′ and R″ are independently selected from C₁ to C₁₆ alkyl, C₆ to C₂₄ aryl, C₃ to C₂₄ heteroaryl;
R³ or R⁴ has the general formula 2: 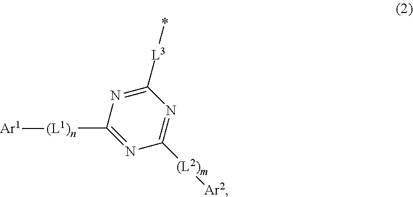
wherein
the asterisk symbol “*” represents the binding position of the group R³ or R⁴;
L¹, L² and L³ are independently selected from substituted or unsubstituted C₆ to C₁₃ arylene or substituted or unsubstituted C₃ to C₁₂ heteroarylene, wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₁₈ aryl, perhalogenated C₆ to C₁₈ aryl, C₃ to C₂₄ heteroaryl, CN, halogen;
n is 1 or 0;
m is 1 or 0;
Ar¹ is selected from substituted or unsubstituted C₅ to C₂₄ heteroaryl,
wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR′,
wherein R′ and R″ are independently selected from C₁ to C₁₆ alkyl, C₆ to C₂₄ aryl, C₃ to C₂₄ heteroaryl; and
wherein Ar¹ is free of a spiro-group;
Ar² has the general formula 3: 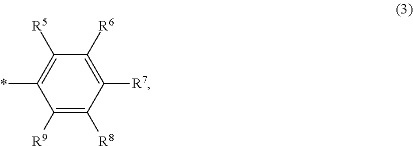
wherein the asterisk symbol “*” represents the binding position of the group Ar² to the moiety L²;
R⁵ to R⁹ are independently H, D, CN, halogen, C₁ to C₁₂ alkyl, C₁ to C₁₂ alkoxy, substituted or unsubstituted C₆ to C₁₂ aryl, substituted or unsubstituted C₃ to C₁₂ heteroaryl,
wherein the substituted aryl ring or substituted heteroaryl ring is substituted with one or more C₁ to C₁₂ alkyl, C₆ to C₁₂ aryl or C₃ to C₁₂ heteroaryl groups, wherein the substituents are bonded by a single bond to the substituted aryl ring or substituted heteroaryl ring;
or
Ar² is selected from a fused ring system comprising one to four substituted or unsubstituted 6 membered aryl rings and zero to two substituted or unsubstituted 5 to 7 membered heteroaryl rings,
wherein the substituted aryl ring is substituted with C₁ to C₁₂ alkyl groups and the substituted heteroaryl ring is substituted with one or more C₁ to C₁₂ alkyl, C₆ to C₁₂ aryl or C₃ to C₁₂ heteroaryl groups;
with the provision that:
i) R³ is formula 2 and R⁴ is selected from C₁ to C₁₂ alkyl, substituted or unsubstituted C₆ to C₁₈ aryl, substituted or unsubstituted C₃ to C₂₄ heteroaryl,
wherein the substituents are selected from C₁ to C₁₂ alkyl, perhalogenated C₁ to C₁₂ alkyl, C₁ to C₁₂ alkoxy, perhalogenated C₁ to C₁₂ alkoxy, C₆ to C₂₄ aryl, perhalogenated C₆ to C₂₄ aryl, C₃ to C₂₄ heteroaryl, CN, halogen, P(═O)R′R″, C(═O)R′ or C(═O)OR
wherein R′ and R″ are independently selected from C₁ to C₁₆ alkyl, C₆ to C₂₄ aryl, C₃ to C₂₄ heteroaryl;
or
ii) R⁴ is formula 2 and R³ is H or D; and
b) at least one organic metal complex, wherein the metal of the organic metal complex is selected from the group comprising alkali, alkaline earth or rare earth metal.

2. The composition according to claim 1, wherein the C₅ to C₂₄ heteroaryl of the substituted or unsubstituted C₅ to C₂₄ heteroaryl of Ar¹ or the substituted or unsubstituted C₅ to C₂₄ heteroaryl of Ar¹ comprises:
at least one substituted or unsubstituted heteroaryl ring,
at least one substituted or unsubstituted heteroaryl ring and at least one substituted or unsubstituted non-heteroaryl ring,
at least two substituted or unsubstituted heteroaryl rings and at least one substituted or unsubstituted non-heteroaryl ring,
at least three substituted or unsubstituted heteroaryl rings and at least one substituted or unsubstituted non-heteroaryl ring,
at least one substituted or unsubstituted heteroaryl ring and at least two substituted or unsubstituted non-heteroaryl rings, or
the substituted or unsubstituted C₅ to C₂₄ heteroaryl are fused rings.

3. The composition according to claim 1, wherein the metal of the organic metal complex is selected from the group comprising alkali, alkaline earth metal, lithium, magnesium or calcium.

4. The composition according to claim 1, wherein the organic metal complex comprises at least one ligand, wherein the ligand is selected from a quinolate or borate group.

5. The composition according to claim 1, wherein R¹ and R² are independently selected from H, D, unsubstituted C₆ to C₁₈ aryl, or unsubstituted C₃ to C₂₄ heteroaryl.

6. The composition according to claim 1, wherein Ar¹ is selected from substituted or unsubstituted C₅ to C₂₄ heteroaryl comprising at least one heteroatom selected from O, S, Se or N.

7. The composition according to claim 1, wherein Ar¹ has the general formula 4: 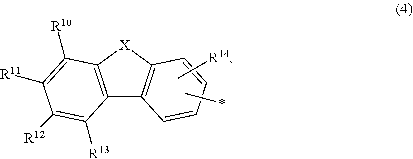
wherein the asterisk symbol “*” represents the binding position of the group Ar¹ to the moiety L¹;
X is selected from O, S, Se or NR¹⁵,
wherein R¹⁵ is independently selected from C₁ to C₁₂ alkyl, perhalogenated C₁ to C₁₂ alkyl, C₆ to C₂₄ aryl, perhalogenated C₆ to C₂₄ aryl, C₃ to C₂₄ heteroaryl;
R¹⁰ to R¹⁴ are independently selected from H, D, CN, halogen, C₁ to C₁₂ alkyl, C₁ to C₁₂ alkoxy, C₆ to C₁₈ aryl or C₃ to C₂₄ heteroaryl.

8. The composition according to claim 7, wherein X of general formula 4 is selected from O, S or NR¹⁵.

9. The composition according claim 1, wherein Ar¹ is selected from the group comprising D1 to D16:

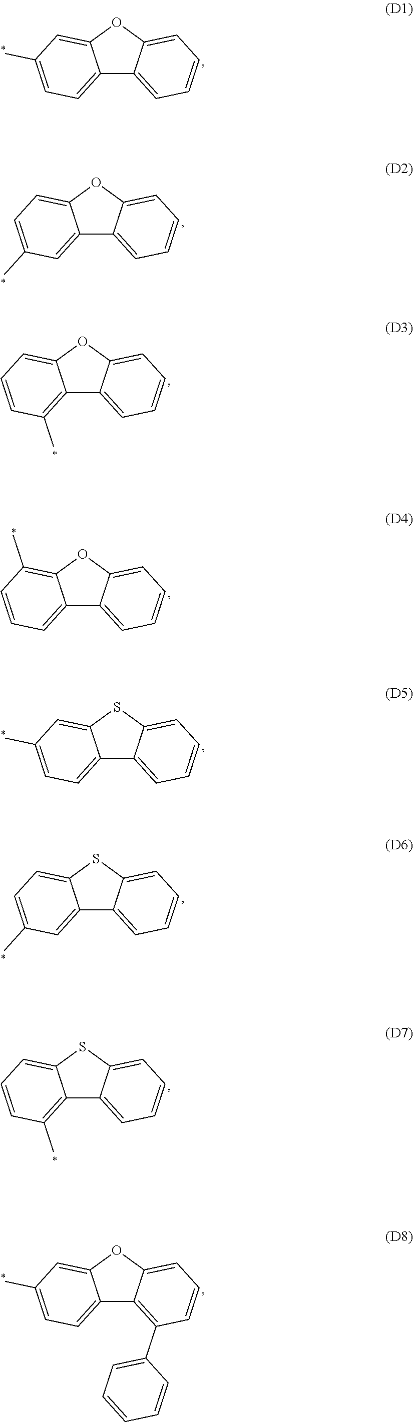

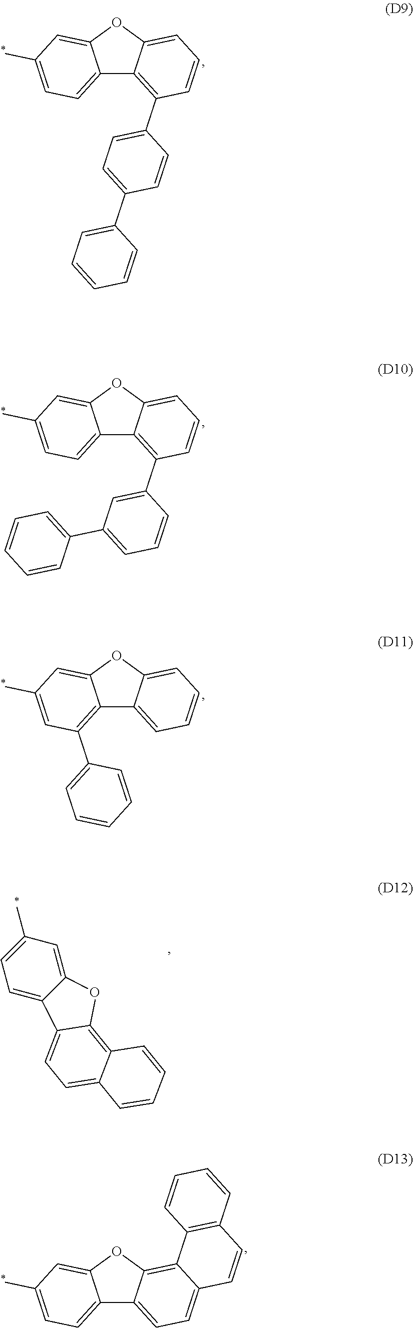 [SMILES: *c1ccc2c(c1)oc1cccc(-c3ccc(-c4ccccc4)cc3)c12 ; *c1ccc2c(c1)oc1cccc(-c3cccc(-c4ccccc4)c3)c12 ; *c1cc(-c2ccccc2)c2c(c1)oc1ccccc12 ; *c1ccc2c(c1)oc1c3ccccc3ccc21 ; *c1ccc2c(c1)oc1c2ccc2ccc3ccccc3c21]

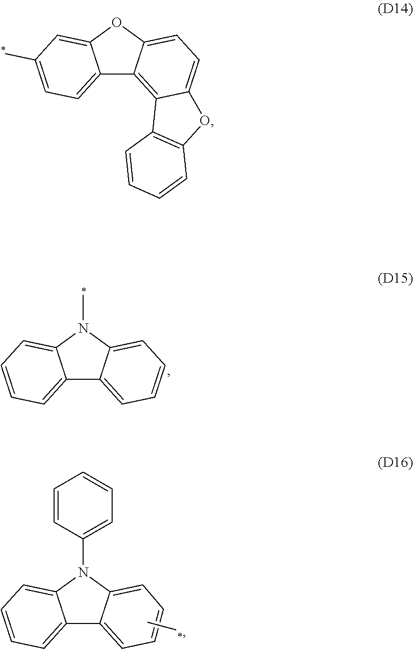

10. The composition according to claim 1, wherein Ar² is selected from the group comprising E1 to E44:

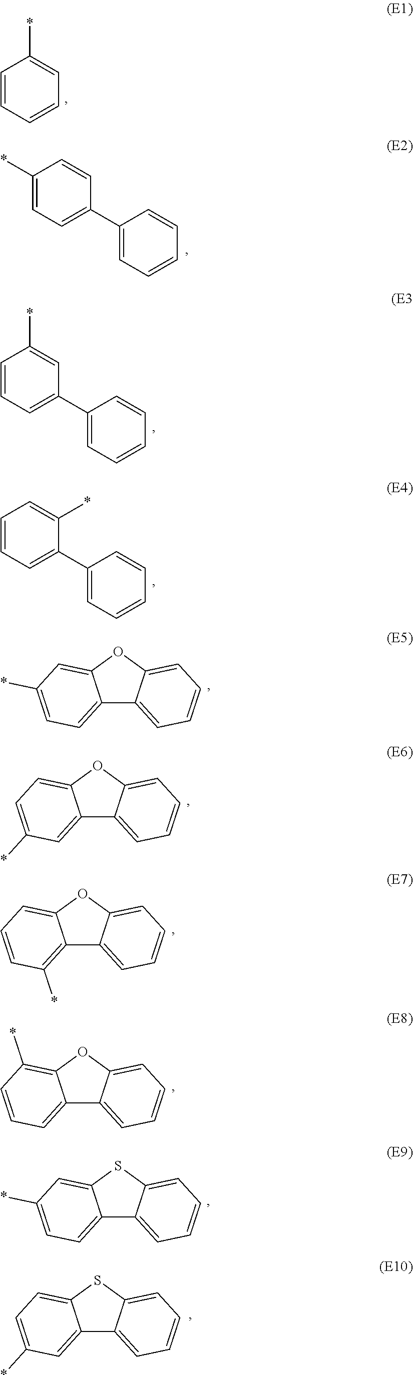

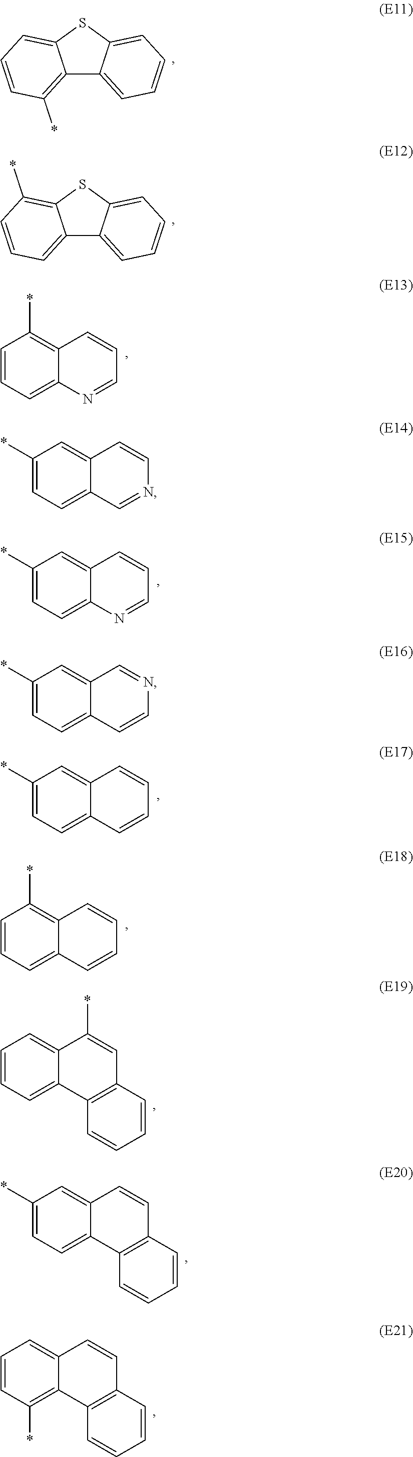

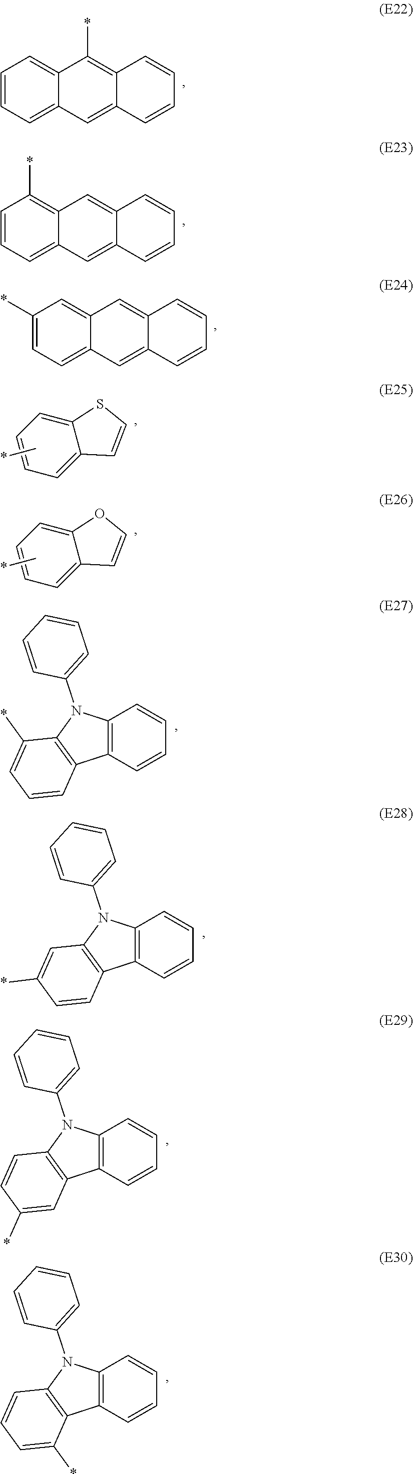

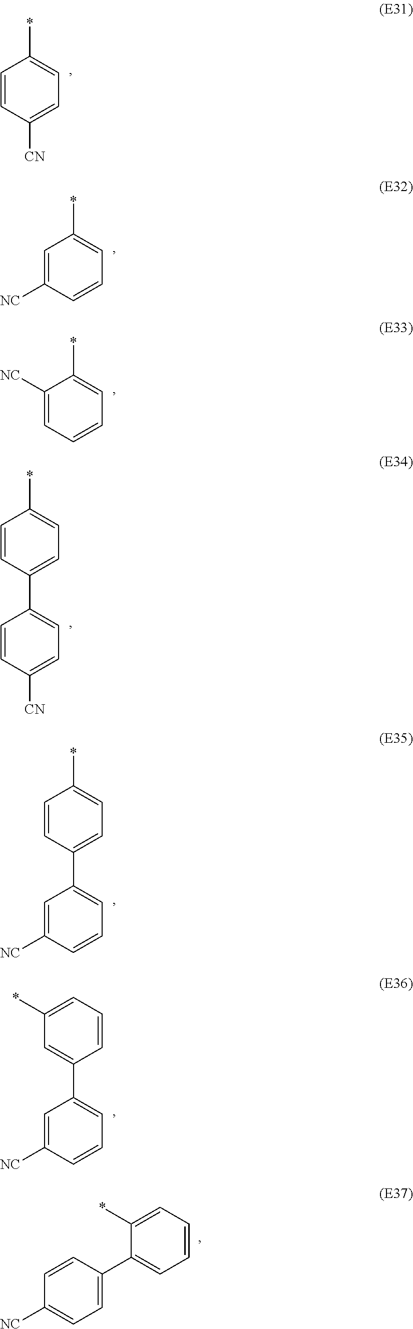

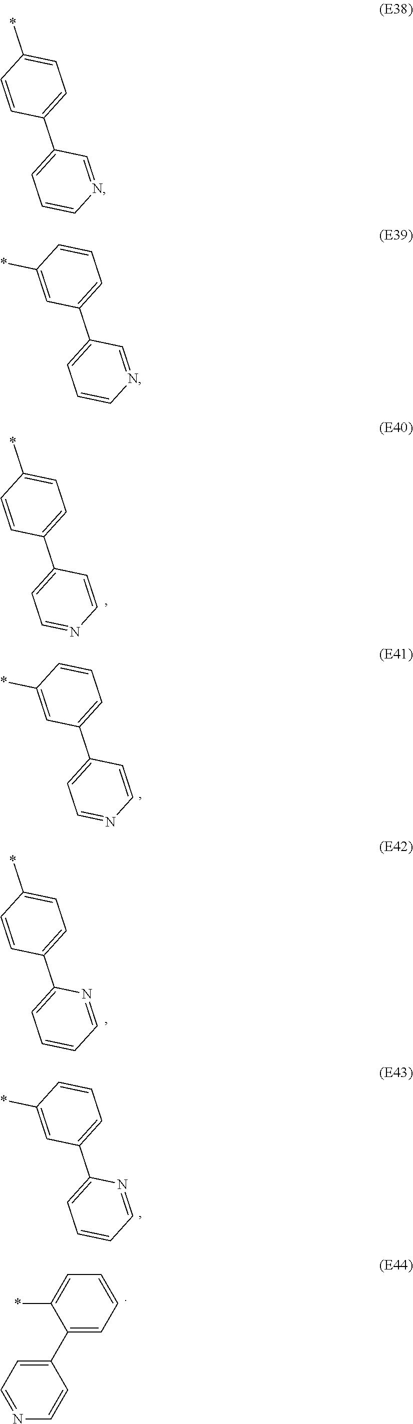

11. The composition according to claim 1, wherein
L¹, L² and/or L³ are independently selected from the group comprising F1 to F11:

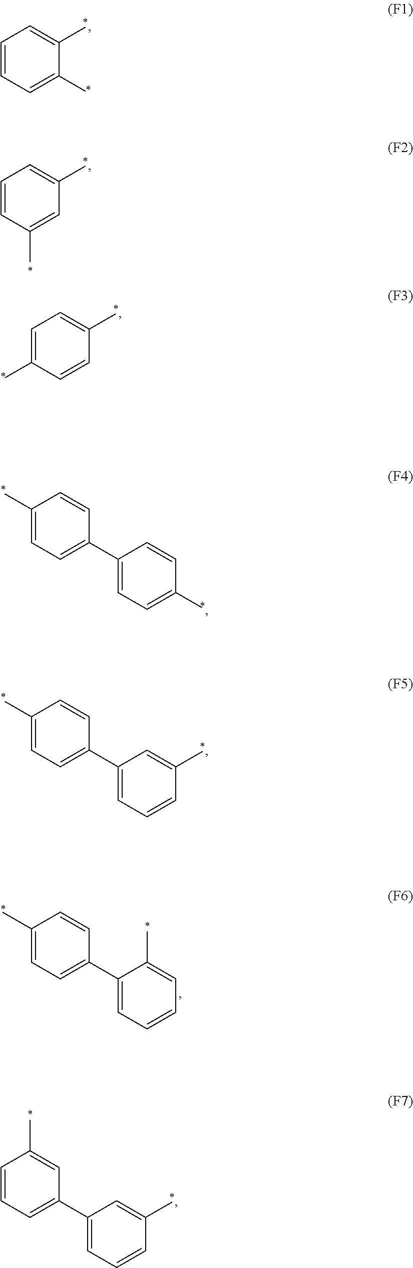

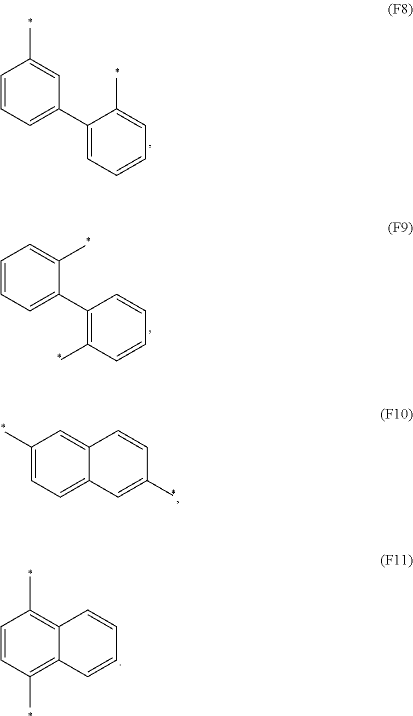

12. The composition according to claim 1, wherein the compounds of formula 1 are selected from the group comprising G1 to G31:

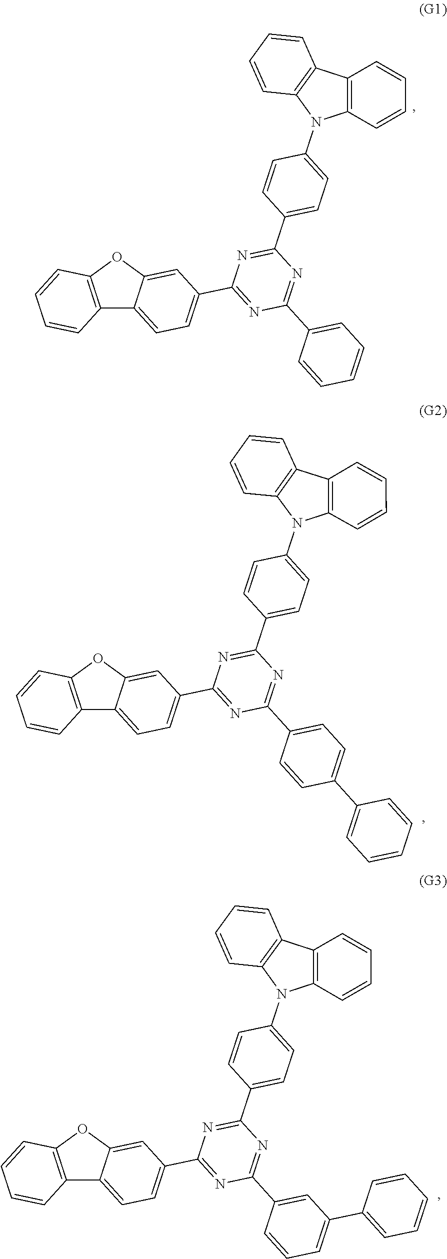 [SMILES: c1ccc(-c2nc(-c3ccc(-n4c5ccccc5c5ccccc54)cc3)nc(-c3ccc4c(c3)oc3ccccc34)n2)cc1 ; c1ccc(-c2ccc(-c3nc(-c4ccc(-n5c6ccccc6c6ccccc65)cc4)nc(-c4ccc5c(c4)oc4ccccc45)n3)cc2)cc1 ; c1ccc(-c2cccc(-c3nc(-c4ccc(-n5c6ccccc6c6ccccc65)cc4)nc(-c4ccc5c(c4)oc4ccccc45)n3)c2)cc1]

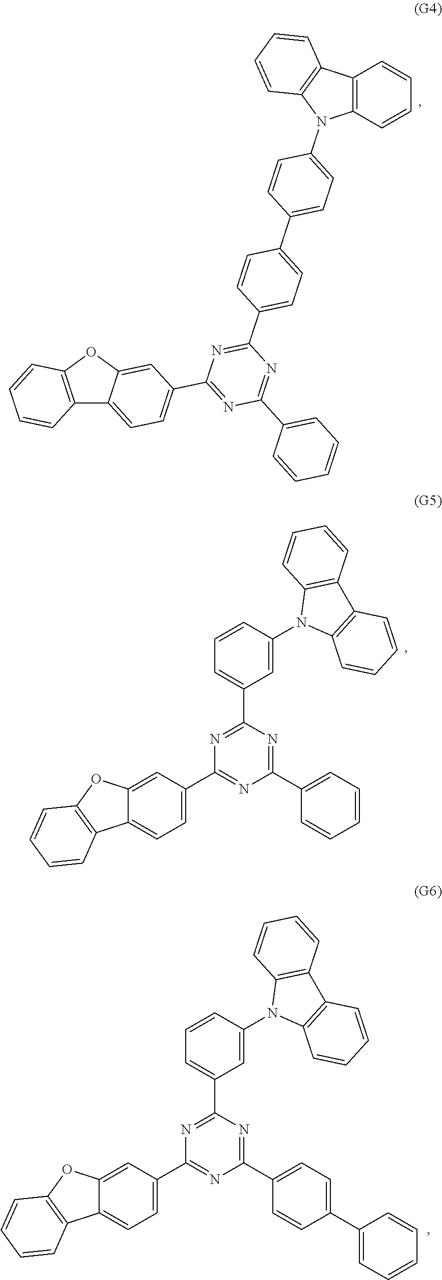 [SMILES: c1ccc(-c2nc(-c3ccc(-c4ccc(-n5c6ccccc6c6ccccc65)cc4)cc3)nc(-c3ccc4c(c3)oc3ccccc34)n2)cc1 ; c1ccc(-c2nc(-c3cccc(-n4c5ccccc5c5ccccc54)c3)nc(-c3ccc4c(c3)oc3ccccc34)n2)cc1 ; c1ccc(-c2ccc(-c3nc(-c4cccc(-n5c6ccccc6c6ccccc65)c4)nc(-c4ccc5c(c4)oc4ccccc45)n3)cc2)cc1]

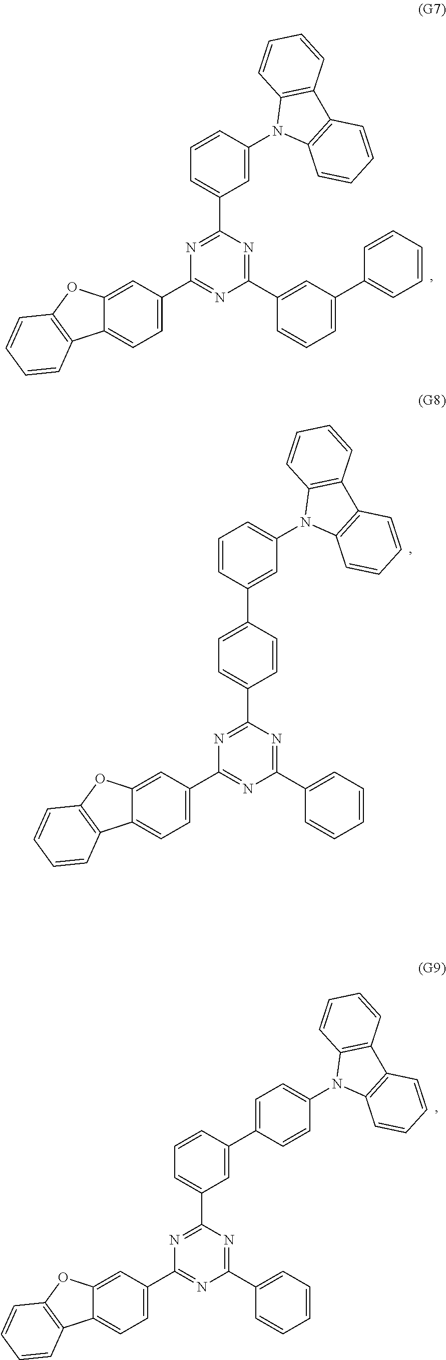 [SMILES: c1ccc(-c2cccc(-c3nc(-c4cccc(-n5c6ccccc6c6ccccc65)c4)nc(-c4ccc5c(c4)oc4ccccc45)n3)c2)cc1 ; c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2nc(-c3cccc(-c4ccc(-n5c6ccccc6c6ccccc65)cc4)c3)nc(-c3ccc4c(c3)oc3ccccc34)n2)cc1]

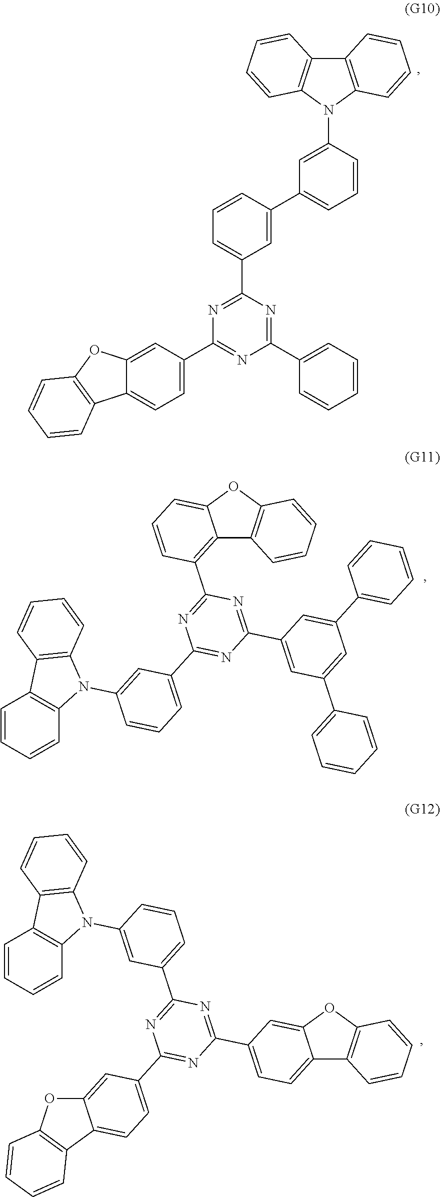 [SMILES: c1ccc(-c2nc(-c3cccc(-c4cccc(-n5c6ccccc6c6ccccc65)c4)c3)nc(-c3ccc4c(c3)oc3ccccc34)n2)cc1 ; c1ccc(-c2cc(-c3ccccc3)cc(-c3nc(-c4cccc(-n5c6ccccc6c6ccccc65)c4)nc(-c4cccc5oc6ccccc6c45)n3)c2)cc1 ; c1cc(-c2nc(-c3ccc4c(c3)oc3ccccc34)nc(-c3ccc4c(c3)oc3ccccc34)n2)cc(-n2c3ccccc3c3ccccc32)c1]

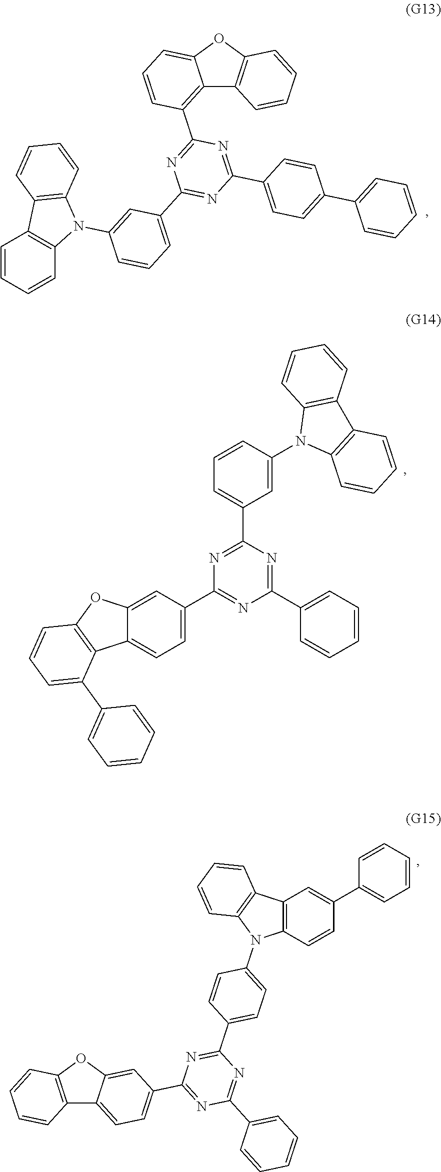 [SMILES: c1ccc(-c2ccc(-c3nc(-c4cccc(-n5c6ccccc6c6ccccc65)c4)nc(-c4cccc5oc6ccccc6c45)n3)cc2)cc1 ; c1ccc(-c2nc(-c3cccc(-n4c5ccccc5c5ccccc54)c3)nc(-c3ccc4c(c3)oc3cccc(-c5ccccc5)c34)n2)cc1 ; c1ccc(-c2ccc3c(c2)c2ccccc2n3-c2ccc(-c3nc(-c4ccccc4)nc(-c4ccc5c(c4)oc4ccccc45)n3)cc2)cc1]

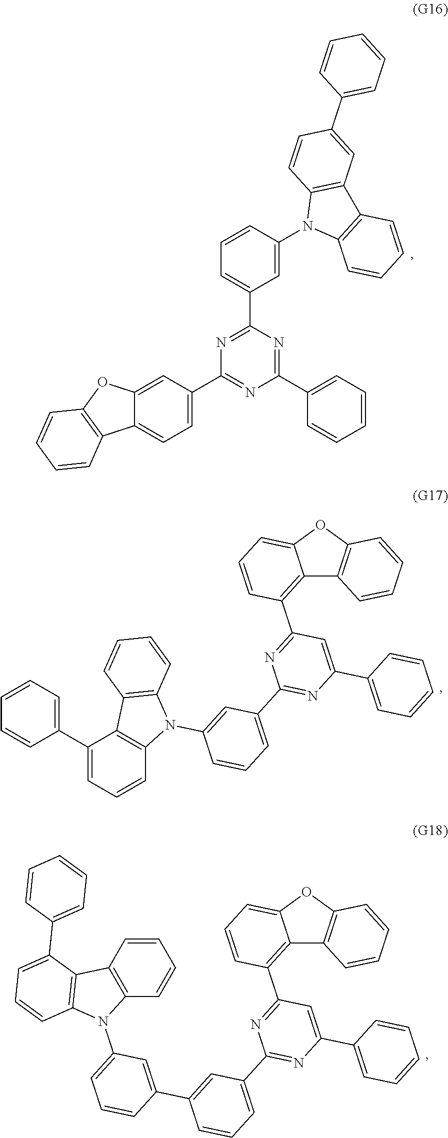 [SMILES: c1ccc(-c2ccc3c(c2)c2ccccc2n3-c2cccc(-c3nc(-c4ccccc4)nc(-c4ccc5c(c4)oc4ccccc45)n3)c2)cc1 ; c1ccc(-c2cc(-c3cccc4oc5ccccc5c34)nc(-c3cccc(-n4c5ccccc5c5c(-c6ccccc6)cccc54)c3)n2)cc1 ; c1ccc(-c2cc(-c3cccc4oc5ccccc5c34)nc(-c3cccc(-c4cccc(-n5c6ccccc6c6c(-c7ccccc7)cccc65)c4)c3)n2)cc1]

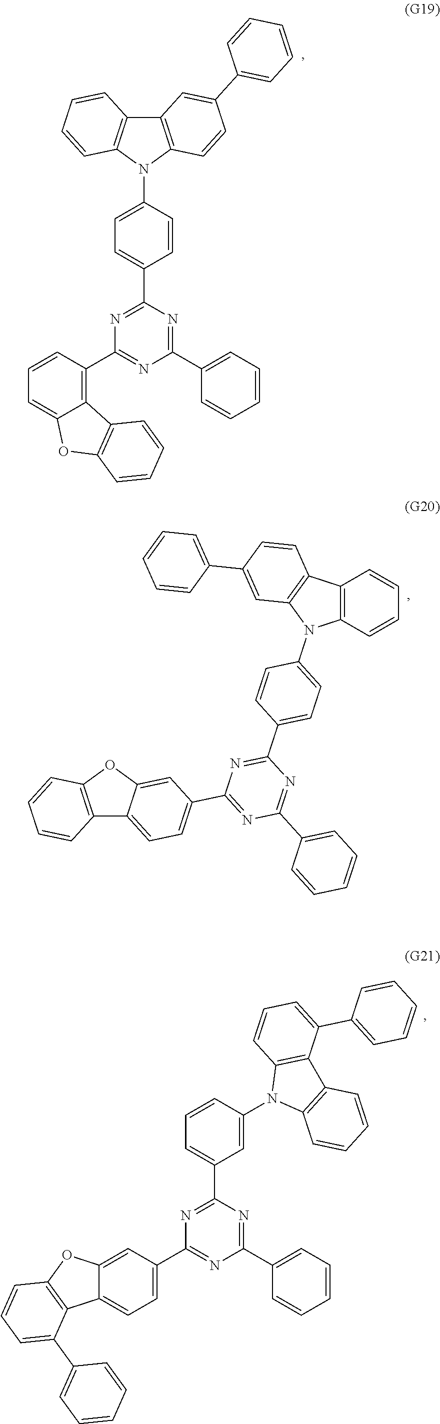 [SMILES: c1ccc(-c2ccc3c(c2)c2ccccc2n3-c2ccc(-c3nc(-c4ccccc4)nc(-c4cccc5oc6ccccc6c45)n3)cc2)cc1 ; c1ccc(-c2ccc3c4ccccc4n(-c4ccc(-c5nc(-c6ccccc6)nc(-c6ccc7c(c6)oc6ccccc67)n5)cc4)c3c2)cc1 ; c1ccc(-c2nc(-c3cccc(-n4c5ccccc5c5c(-c6ccccc6)cccc54)c3)nc(-c3ccc4c(c3)oc3cccc(-c5ccccc5)c34)n2)cc1]

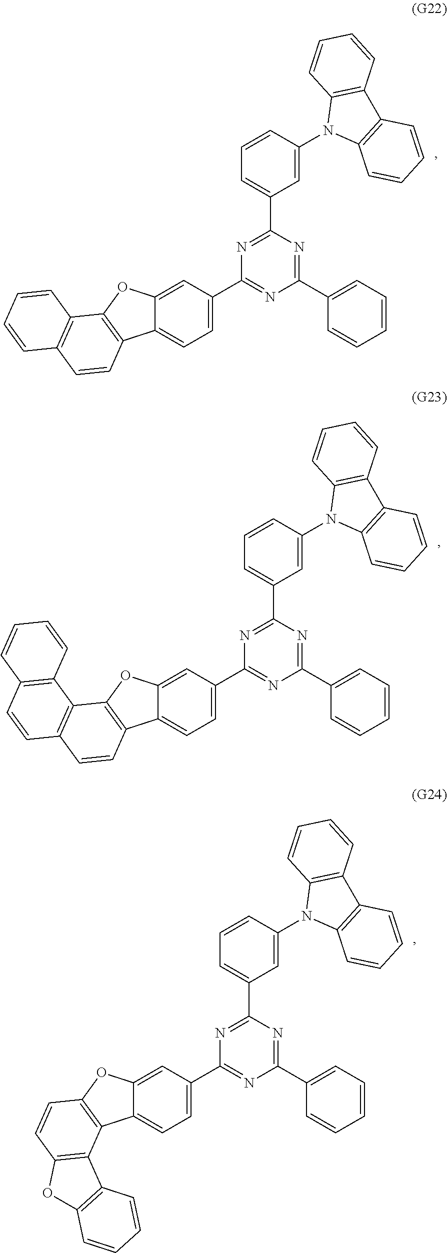 [SMILES: c1ccc(-c2nc(-c3cccc(-n4c5ccccc5c5ccccc54)c3)nc(-c3ccc4c(c3)oc3c5ccccc5ccc43)n2)cc1 ; c1ccc(-c2nc(-c3cccc(-n4c5ccccc5c5ccccc54)c3)nc(-c3ccc4c(c3)oc3c4ccc4ccc5ccccc5c43)n2)cc1 ; c1ccc(-c2nc(-c3cccc(-n4c5ccccc5c5ccccc54)c3)nc(-c3ccc4c(c3)oc3ccc5oc6ccccc6c5c34)n2)cc1]

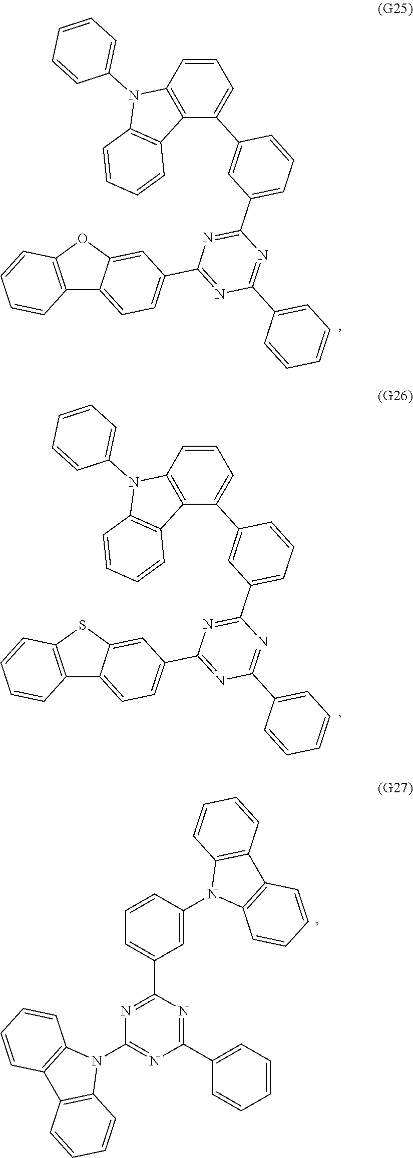 [SMILES: c1ccc(-c2nc(-c3cccc(-c4cccc5c4c4ccccc4n5-c4ccccc4)c3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)sc3ccccc34)n2)cc1 ; c1ccc(-c2nc(-c3cccc(-n4c5ccccc5c5ccccc54)c3)nc(-n3c4ccccc4c4ccccc43)n2)cc1]

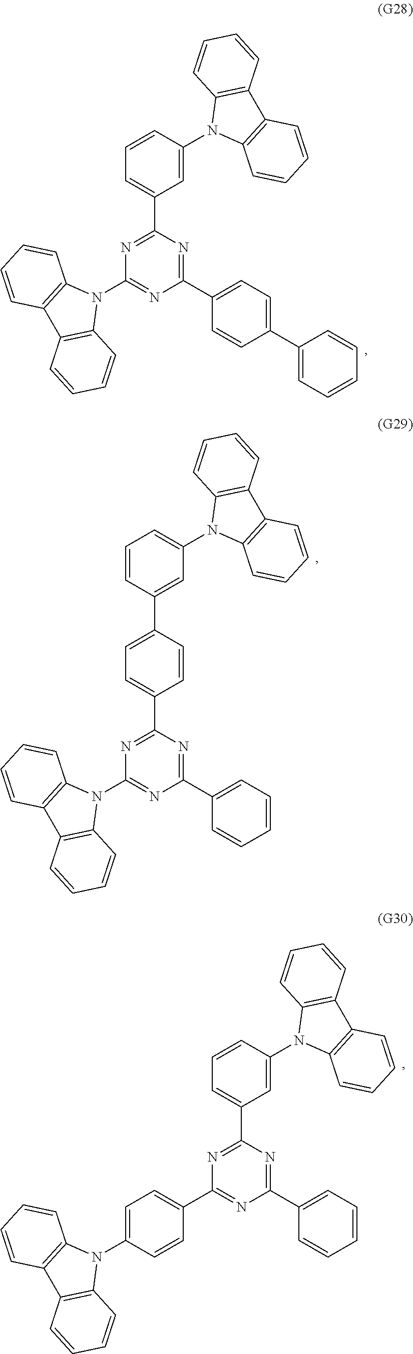 [SMILES: c1ccc(-c2ccc(-c3nc(-c4cccc(-n5c6ccccc6c6ccccc65)c4)nc(-n4c5ccccc5c5ccccc54)n3)cc2)cc1 ; c1ccc(-c2nc(-c3ccc(-c4cccc(-n5c6ccccc6c6ccccc65)c4)cc3)nc(-n3c4ccccc4c4ccccc43)n2)cc1 ; c1ccc(-c2nc(-c3ccc(-n4c5ccccc5c5ccccc54)cc3)nc(-c3cccc(-n4c5ccccc5c5ccccc54)c3)n2)cc1]

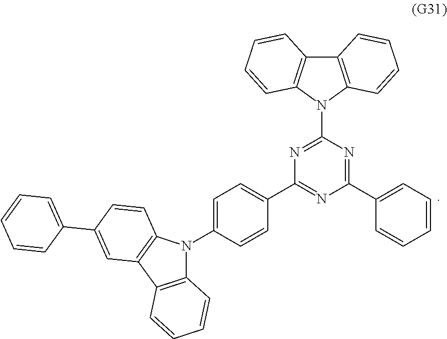 [SMILES: c1ccc(-c2ccc3c(c2)c2ccccc2n3-c2ccc(-c3nc(-c4ccccc4)nc(-n4c5ccccc5c5ccccc54)n3)cc2)cc1]

13. An organic semiconductor layer comprising at least one composition according to claim 1.

14. An organic electronic device comprising at least one organic semiconductor layer according to claim 13.

15. The organic electronic device according to claim 14, further comprising at least one anode and at least one cathode.

16. The organic electronic device according to 14, wherein the organic electronic device is selected front she group comprising a light emitting device, thin film transistor, a battery, a display device or a photovoltaic cell.

17. The composition according to claim 1, wherein Ar¹ has the general formula 4: 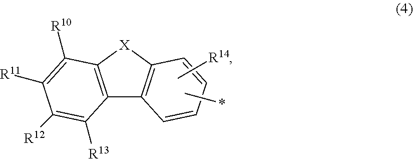
wherein the asterisk symbol “*” represents the binding position of the group Ar¹ to the moiety L¹;
X is selected from O, S, Se or NR¹⁵,
wherein R¹⁵ is independently selected from C₁ to C₁₂ alkyl, perhalogenated C₁ to C₁₂ alkyl, C₆ to C₂₄ aryl, perhalogenated C₆ to C₂₄ aryl, C₃ to C₂₄ heteroaryl;
R¹⁰ to R¹⁴ are independently selected from H, D, CN, halogen, C₁ to C₁₂ alkyl, C₁ to C₁₂ alkoxy, C₆ to C₁₈ aryl or C₃ to C₂₄ heteroaryl;
and at least one of R¹⁰ and R¹¹, R¹¹ and R¹² or R¹² and R¹³ form a 5 to 7 membered substituted or unsubstituted aryl ring, or a 5 to 7 membered substituted or unsubstituted heteroaryl ring,
wherein the substituents are selected from C₁ to C₁₂ alkyl, C₆ to C₁₈ aryl or C₃ to C₂₄ heteroaryl groups.

18. The composition according to claim 1, wherein Ar¹ has the general formula 4: 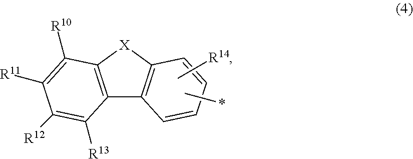
wherein the asterisk symbol “*” represents the binding position of the group Ar¹ to the moiety L¹;
X is selected from O, S, Se or NR¹⁵,
wherein R¹⁵ is independently selected from C₁ to C₁₂ alkyl, perhalogenated C₁ to C₁₂ alkyl, C₆ to C₂₄ aryl, perhalogenated C₆ to C₂₄ aryl, C₃ to C₂₄ heteroaryl;
R¹⁰ to R¹⁴ are independently selected from H, D, CN, halogen, C₁ to C₁₂ alkyl, C₁ to C₁₂ alkoxy, C₆ to C₁₈ aryl or C₃ to C₂₄ heteroaryl;
wherein preferably R¹⁰ and R¹¹, R¹¹ and R¹² or R¹² and R¹³ form a 5 to 7 membered substituted or unsubstituted aryl ring, or a 5 to 7 membered substituted or unsubstituted heteroaryl ring, and in addition at least one of R¹⁰ and R¹¹, R¹¹ and R¹² or R¹² and R¹³ form two fused 5 to 7 membered substituted or unsubstituted aryl rings, or two fused 5 to 7 membered substituted or unsubstituted heteroaryl rings,
wherein the substituents are selected from C₁ to C₁₂ alkyl, C₆ to C₁₈ aryl or C₃ to C₂₄ heteroaryl groups.

19. The composition according to claim 1, wherein for L¹ n is 1 and for L² m is 0, L¹ is selected from F1 to F11.

20. The composition according to claim 1, wherein for L¹ n is 0 and for L² m is 1, L² is selected from F1 to F11.

21. The composition according to claim 1, wherein for L¹ n is 0 and for L² m is 0 and L³ is selected from F1 to F11.
